(19) United States
(12) Patent Application Publication
(10) Pub. No.: US 2024/0094215 A1
(43) Pub. Date: Mar. 21, 2024

(54) CHARACTERIZING ACCESSIBILITY OF MACROMOLECULE STRUCTURES

(71) Applicant: NAUTILUS SUBSIDIARY, INC., Seattle, WA (US)

(72) Inventors: Taryn GILLIES, Philadelphia, PA (US); James Henry JOLY, San Mateo, CA (US); Jarrett D. EGERTSON, Rancho Palos Verdes, CA (US)

(21) Appl. No.: 18/466,641

(22) Filed: Sep. 13, 2023

Related U.S. Application Data
(60) Provisional application No. 63/375,833, filed on Sep. 15, 2022.

(51) Int. Cl. G01N 33/68

(52) U.S. Cl. CPC G01N 33/6803

(57) ABSTRACT

A method including contacting an array of macromolecules with a plurality of different assay reagents, wherein individual addresses of the array are attached to single macromolecules, and the macromolecule comprising a plurality of different reactive sites; detecting reaction of the array of macromolecules with the different assay reagents at single-molecule resolution; determining a first reaction extent comprising the fraction of the individual addresses observed to react with a first assay reagent; determining a second reaction extent comprising the fraction of the individual addresses observed to react with a second assay reagent; determining an observed double reaction extent comprising the fraction of the individual addresses observed to react with both the first and second assay reagents; determining an expected double reaction extent from the first and second reaction extents; and determining accessibility of a reactive site of the macromolecules based on a comparison of the observed and expected double reaction extents.

### CROSS-REFERENCE TO RELATED APPLICATIONS

This application claims priority to U.S. Provisional Application No. 63/375,833, filed on Sep. 15, 2022, which is incorporated herein by reference in its entirety.

### BACKGROUND

Proteins are polymers formed from sequences of amino acids, amino acids being the monomeric subunits of the polymer. In biological systems, the sequence of amino acids for a given protein is encoded by the DNA sequence of its gene. Proteins are synthesized in biological systems via processes that transcribe and translate the DNA sequences of the genes to produce the amino acid sequences that constitute the proteins. The collection of different proteins synthesized from an organism's full collection of genes (i.e. the genome of the organism) constitutes its proteome. The proteins in a proteome have a wide range of different amino acid sequences and this diversity plays a significant role in the diversity of characteristics and functions of the organism. The functions of the proteins derive from their structures and, as such, knowledge of a protein's structure provides valuable insight into understanding the characteristics and health of the organism from which the protein is derived.

Although knowing a protein's sequence of amino acids can provide useful hints as to the function of the protein, a far greater wealth of information can be gleaned from knowledge of the three-dimensional shape into which the protein polymer folds. Atomic-resolution structures are the gold standard. Unfortunately, atomic-scale methods are expensive and low throughput, and, to date, three-dimensional structures are known for only a small fraction of all known proteins. Moreover, empirical methods that provide structures at atomic resolution do so by maintaining proteins in somewhat contrived conditions. For example, many protein structures have been obtained from techniques that observe proteins in a crystalized state, which although accepted as a realistic conformational state, does not capture the wide range of conformational states assumed by any given protein in its native milieu. Advances have been made in predicting the three-dimensional fold of proteins from their amino acid sequences. However, structures for a large number of proteins have not been successfully solved using these predictive methods. Moreover, the predictive algorithms have been trained and validated based on empirical data acquired from crystalized proteins. Thus, there is a need for methods and systems to determine protein three-dimensional structures, especially at proteome scales. The present disclosure satisfies this need and provides other advantages as well.

### SUMMARY

The present disclosure provides a method of determining accessibility of macromolecule structures. The method can include steps of (a) contacting an array of macromolecules with a plurality of different assay reagents, wherein individual addresses of the array are each attached to a single macromolecule, the macromolecule having a plurality of different reactive sites, and wherein the different assay reagents are different with respect to specificity for the reactive sites; (b) detecting reaction of the array of macromolecules with the different assay reagents, whereby the individual addresses of the array are resolved, and whereby the different assay reagents are resolved; (c) determining a first observed reaction extent comprising the fraction of the individual addresses having a first reactive site that is observed to react with a first assay reagent of the plurality of different assay reagents in step (b); (d) providing an expected reaction extent including the extent to which the assay reagent reacts with a candidate macromolecule having the same molecular structure as the macromolecules at the fraction of the individual addresses observed to react with the first assay reagent in step (b); and (e) determining accessibility of the first reactive site of the macromolecules based on a comparison of the observed reaction extent and the expected reaction extent. Optionally, the assay reagents are affinity reagents, the reaction is binding of the affinity reagents to macromolecules of the array, the reactive sites are epitopes and the reaction extents are binding extents.

The present disclosure provides a method of detecting a plurality of macromolecules at single-molecule resolution and evaluating the dynamics of the plurality to determine accessibility of macromolecule structures. The method can include the steps of (a) contacting an array of macromolecules with a plurality of different assay reagents, wherein individual addresses of the array are each attached to a single macromolecule, the macromolecule including a plurality of different reactive sites, and wherein the different assay reagents are different with respect to specificity for the reactive sites; (b) detecting reaction of the array of macromolecules with the different assay reagents, whereby the individual addresses of the array are resolved, and whereby the different assay reagents are resolved; (c) determining a first observed reaction extent including the fraction of the individual addresses observed to react with a first assay reagent in step (b); (d) determining a second observed reaction extent including the fraction of the individual addresses observed to react with a second assay reagent in step (b); (e) determining an observed double reaction extent including the fraction of the individual addresses observed to react with both the first assay reagent and the second assay reagent in step (b); (f) determining an expected double reaction extent from the first observed reaction extent and the second observed reaction extent; and (g) determining accessibility of a first reactive site of the plurality of different reactive sites based on a comparison of the observed double reaction extent and the expected double reaction extent. Optionally, the assay reagents are affinity reagents, the reaction is binding of the affinity reagents to macromolecules of the array, the reactive sites are epitopes and the reaction extents are binding extents.

Also provided is a system for determining accessibility of macromolecule structures. The system can include (a) a detector configured to acquire signals from an array of macromolecules contacted with a plurality of different assay reagents, wherein individual addresses of the array are each attached to a single macromolecule, the macromolecule having a plurality of different reactive sites, and wherein the different assay reagents are different with respect to specificity for the reactive sites; (b) a computer processor configured to receive signals from the detector, wherein the signals are resolved with respect to the individual addresses of the array, and wherein the signals are resolved with respect to the different assay reagents, and: (i) determine a first observed reaction extent from the received signals, the first observed reaction extent configured as the fraction of the individual addresses observed to react with a first assay reagent, (ii) determine a second observed reaction extent from the received signals, the second observed reaction extent configured as the fraction of the individual addresses observed to react with a second assay reagent, (iii) determine an observed double reaction extent from the received signals, the second observed reaction extent configured as the fraction of the individual addresses observed to react with both the first assay reagent and the second assay reagent, (iv) determine an expected double reaction extent from the first observed reaction extent and the second observed reaction extent; and (v) determine accessibility of a first reactive site of the plurality of different reactive sites based on a comparison of the observed double reaction extent and the expected double reaction extent. Optionally, the assay reagents are affinity reagents, the reaction is binding of the affinity reagents to macromolecules of the array, the reactive sites are epitopes and the reaction extents are binding extents.

The present disclosure further provides a method of determining accessibility of macromolecule structures, including steps of (a) contacting an array of macromolecules with a plurality of different assay reagents, wherein individual addresses of the array are each attached to a single macromolecule, the macromolecule comprising a plurality of different reactive sites, and wherein the different assay reagents are different with respect to specificity for the reactive sites; (b) detecting reaction outcomes for individual addresses in the array with each of the different assay reagents, whereby the individual addresses of the array are resolved, whereby the different assay reagents are resolved, and wherein a collection of reaction outcomes for each of the addresses forms an empirical outcome profile; (c) comparing the empirical outcome profile for each of the addresses with a plurality of candidate outcome profiles, each of the candidate outcome profiles including a probability of a given candidate macromolecule reacting with the different assay reagents, thereby identifying a set of addresses having a similar candidate macromolecule; (d) identifying at least two subsets of the addresses having the similar candidate macromolecule, wherein the two subsets include different isoforms of the similar candidate macromolecule; and (e) evaluating reaction outcomes for the different isoforms to determine differential accessibility of a first reactive site in the different isoforms of the similar candidate macromolecule.

In a particular configuration, a method of determining accessibility of macromolecule structures, can include steps of (a) contacting an array of proteins with a plurality of different affinity reagents, wherein individual addresses of the array are each attached to a single protein, the protein comprising a plurality of different epitopes, and wherein the different affinity reagents are different with respect to specificity for the epitopes; (b) detecting reaction outcomes for individual addresses in the array with each of the different affinity reagents, whereby the individual addresses of the array are resolved, whereby the different affinity reagents are resolved, and wherein a collection of reaction outcomes for each of the addresses forms an empirical outcome profile; (c) comparing the empirical outcome profile for each of the addresses with a plurality of candidate outcome profiles, each of the candidate outcome profiles including a probability of a given candidate protein reacting with the different affinity reagents, thereby identifying a set of addresses having a similar candidate protein; (d) identifying at least two subsets of the addresses having the similar candidate protein, wherein the two subsets include different proteoforms of the similar candidate protein; and (e) evaluating reaction outcomes for the different proteoforms to determine differential accessibility of a first epitope in the different isoforms of the similar candidate protein. Optionally, step (e) includes (i) representing the similar candidate protein as a sequence of ordered epitopes, (ii) determining probability that the empirical reaction outcomes correspond to individual epitopes in the sequence of ordered epitopes, and (iii) identifying a region of unlikely non-binding of affinity reagents in the sequence of ordered epitopes.

### INCORPORATION BY REFERENCE

All publications, items of information available on the internet, patents, and patent applications cited in this specification are herein incorporated by reference to the same extent as if each individual publication, item, patent, or patent application was specifically and individually indicated to be incorporated by reference. To the extent publications, items of information available on the internet, patents, or patent applications incorporated by reference contradict the disclosure contained in the specification, the specification is intended to supersede and/or take precedence over any such contradictory material.

### BRIEF DESCRIPTION OF THE DRAWINGS

FIG. 1 shows a simulated protein accessibility plot based on binding rates for epitopes at different positions along the amino acid sequence of the protein.

FIG. 2 shows a workflow from sample preparation to data analysis for a method of identifying proteins.

FIG. 3 shows a computer system that is programmed or otherwise configured to implement a method set forth herein.

FIG. 4A shows the results of simulating decoding of addresses having full length PARP1.

FIG. 4B shows the results of simulating decoding of addresses having cleaved PARP1.

FIG. 4C shows the combined results of FIGS. 4A and 4B.

### DETAILED DESCRIPTION

The present disclosure provides methods, systems and compositions that can be used to determine accessibility of macromolecules to assay reagents. Localized accessibility of regions within a macromolecule can be determined, for example, in order to characterize structural features of the macromolecules. Exemplary structural features of a macromolecule that can be characterized include, for example, the three-dimensional structure of the macromolecule, the presence of chemically modified sites on the macromolecule, insertions of a subunit or sequence of subunits in the macromolecule, or deletions of a subunit or sequence of subunits in the macromolecule. For example, the three-dimensional structure of proteins can be characterized by distinguishing amino acids that are readily accessible to assay reagents, due to their location on the surface of the protein, from amino acids that are inaccessible to the reagents due to their location within the globular structure of the protein. In another example, the presence and absence of post-translational modifications at particular sites in proteins can be determined based on differential accessibility of those sites to assay reagents that are differentially reactive to the sites in modified and unmodified forms. In a further example, insertions or deletions in the amino acid sequences of proteins can be characterized by distinguishing amino acids that are present and accessible to assay reagents from amino acids that are inaccessible to the assay reagents, due to their absence in the protein.

An example of assaying proteins using affinity reagents can be illustrative. An array having 1000 copies of Protein X can be assayed for binding to five different affinity reagents identified as AR1 through AR5. In this example, the array is in a single-molecule format, whereby each copy of Protein X is attached to an address on the array and each of those addresses is attached to only one protein. The five affinity reagents recognize different epitopes, the different epitopes being located at different positions along the amino acid sequence of Protein X. Assume that the following results are observed: AR1 binds 500 of the 1000 addresses (i.e. an observed binding rate of 0.5), AR2 binds 500 of the 1000 addresses (i.e. an observed binding rate of 0.5), AR3 binds 200 of the 1000 addresses (i.e. an observed binding rate of 0.2), AR4 binds 200 of the 1000 addresses (i.e. an observed binding rate of 0.2), and AR5 binds 500 of the 1000 addresses (i.e. an observed binding rate of 0.5). Assume that each of the affinity reagents has an expected binding rate of 0.5 based on control assays performed with Protein X. In this example, the observed binding rates for AR1, AR2 and AR5 match the expected binding rates, thereby indicating that the epitopes are located in regions of Protein X having expected accessibility; however, the epitopes for AR3 and AR4 have lower than expected accessibility as apparent from their observed binding rates being lower than their expected binding rates. The reduced accessibility may indicate a conformational perturbation in the domain of Protein X where the epitopes for AR3 and AR4 reside, the presence of post-translational modifications to the epitopes for AR3 and AR4, or a deletion of the epitopes for AR3 and AR4. Further analyses can be performed to identify the cause for the reduced accessibility. For example, the presence of post-translational moieties can be determined if the epitopes for AR3 and AR4 are known to include target sites for post-translational modifications and if presence of the post-translational modifications is known to inhibit binding of AR3 and AR4. Also, the proteins can be assayed for reaction to probes that are specific for the post-translational moieties.

In the above example, binding results from multiple assays are compared. In alternative configurations of the present methods, binding results can be compared within a single assay. For example, differential accessibility of two reactive sites in a given type of macromolecule can be determined from an assay in which a plurality of macromolecules of the given type are reacted with two different assay reagents, the two different assay reagents having differential specificity for the two reactive sites. Differential accessibility of the two reactive sites can be determined from a comparison of (a) the quantity of macromolecules in the assay that react with one of the assay reagents to (b) the quantity of macromolecules in the assay that react with both of the assay reagents.

An example of assaying proteins using a binding assay can be illustrative. The assay can be performed using an array having 1000 copies of Protein AB, each copy being attached to an individual address in the array. Protein AB has an epitope for affinity reagent A and another epitope for affinity reagent B, the affinity reagents being selective for their respective epitopes. The array is contacted with affinity reagent A, then addresses that bind to affinity reagent A are detected, then the array is contacted with affinity reagent B, and addresses that bind to affinity reagent B are detected. Assume that the following is observed: 250 addresses bind to affinity reagent A, 250 addresses bind to affinity reagent B, and 125 addresses bind to both affinity reagent A and affinity reagent B. Considering the single binding events for affinity reagents A and B, each affinity reagent has a binding rate of 25% (250/1000). Given these binding rates, 6.25% of proteins can be expected to bind both affinity reagents (25%*25%=6.25%). However, the empirical results of the assay indicate that 12.5% of the addresses actually bound both affinity reagents, double the percentage expected. The mismatch in the expected and observed double positive binding rate can be explained by protein accessibility. If only 50% of the 1000 proteins are in an accessible state only 500 are available for affinity reagents to bind. Thus, the empirical observation of 250 binding events for affinity reagents A and B translates into a binding rate of 50%, and the rates of double positive events are expected to be 25%, which aligns with empirical observation of 125/500 available addresses being bound. Protein accessibility can be estimated with the following equation:

$$Accessability = \frac{expected double positive rate}{observed double positive rate}$$

In this exemplary scenario, the equation would indicate 6.25%/12.5%=50% of the proteins have binding sites that are accessible to reagents A and B.

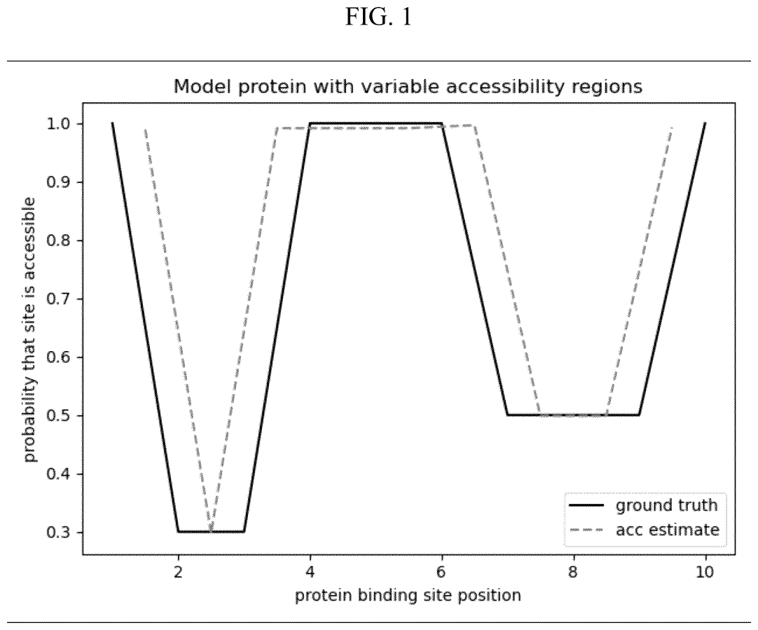

For an assay that uses a larger set of affinity reagents (i.e. a set targeting more than two epitopes in protein AB), this approach can be extended to investigate variable accessibility regions across the protein. For a protein with greater than 3 binding sites, a sliding window can be used to calculate accessibility of reactive site pairs across the protein, using the known probe binding rates to calculate the expected double positive rates for the two probes in the window. An exemplary, diagram of a sliding window analysis is shown in FIG. 1. A similar approach can be applied to other macromolecules using any of a variety of assays as set forth in further detail below.

In particular configurations, accessibility of macromolecule structures can be determined by (a) obtaining empirical reaction outcomes for individual macromolecules with a plurality of different assay reagents; (b) comparing the empirical reaction outcomes with candidate outcomes, wherein the candidate outcomes represent probabilities of a given candidate macromolecule reacting with the different assay reagents; (c) identifying a set of macromolecules having empirical reaction outcomes that are compatible with a particular candidate macromolecule; (d) identifying at least two subsets of the identified macromolecules, the two subsets comprising different isoforms of the particular candidate macromolecule, respectively; and (e) evaluating reaction outcomes to determine differential accessibility of reactive sites in the different isoforms.

Optionally, the macromolecules are proteins, the assay reagents are affinity reagents, the reaction outcomes are outcomes for binding of the assay reagents to the proteins, the candidate outcomes are probabilities of the affinity reagents binding to candidate proteins known or suspected of being among the assayed proteins and the different isoforms are proteoforms of the candidate protein. In this case, steps (a) through (c) of the above method can be performed, for example, as set forth herein below or in US Pat. App. Pub. No. 2023/0114905 A1, or Egertson et al., BioRxiv (2021), DOI: 10.1101/2021.10.11.463967, each of which is incorporated herein by reference. Furthermore, the subsets of the identified protein can be identified, for example, using an unsupervised learning method such as k-means clustering or latent Dirichlet allocation. As a further option, differential accessibility of the reactive sites can be determined by (i) representing the candidate protein as a sequence of ordered epitopes, (ii) determining the probability that the empirical reaction outcomes correspond to individual epitopes in the sequence of ordered epitopes, and (iii) identifying a region of unlikely non-binding of affinity reagents in the sequence of ordered epitopes.

Terms used herein will be understood to take on their ordinary meaning in the relevant art unless specified otherwise. Several terms used herein and their meanings are set forth below.

As used herein, the term “accessibility,” when used in reference to a moiety of a macromolecule or other object in a fluid, refers to exposure of the moiety to the fluid. A moiety can be considered as being accessible to the extent an analyte or reagent in solution is capable of interacting with the moiety, for example, resulting in binding of the analyte or reagent to the moiety, resulting in chemical modification of the moiety by the analyte or reagent, or resulting in chemical modification of the analyte or reagent by the moiety. A moiety can be considered as being inaccessible to the extent an analyte or reagent in solution is incapable of interacting with the moiety. For example, the moiety can be sequestered within a macromolecular structure such that the analyte or reagent is inhibited or the moiety can include a modification (e.g. a post-translational modification) that inhibits the analyte or reagent. Accessibility can be expressed in relative terms, for example, in terms of the size or chemical properties of an analyte or reagent that is used to detect accessibility. In other examples, accessibility can be expressed in terms of relative exposure of different moieties to the same fluid, or in terms of relative exposure of a given moiety in two macromolecules having the same chemical composition. A moiety of a macromolecule can be considered to be inaccessible if it is not present (e.g. a moiety that has been deleted from the macromolecule).

As used herein, the term “address” refers to a location in an array where a particular analyte (e.g. macromolecule, protein, or unique identifier label) is present. An address can contain a single analyte, or it can contain a population of several analytes of the same type (i.e. an ensemble of the analytes). Alternatively, an address can include a population of different analytes. Addresses are typically discrete. Discrete addresses that are adjacent to each other can be contiguous, or they can be separated by interstitial spaces. An array useful herein can have, for example, addresses that are separated by less than 100 microns, 10 microns, 1 micron, 100 nm, 10 nm or less. Alternatively or additionally, an array can have addresses that are separated by at least 10 nm, 100 nm, 1 micron, 10 microns, or 100 microns. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁷, 1×10⁸, 1×10⁹, 1×10¹⁰, 1×10¹¹, 1×10¹², or more addresses.

As used herein, the term “affinity reagent” refers to a molecule or other substance that is capable of specifically or reproducibly binding to an analyte (e.g. protein). An affinity reagent can be larger than, smaller than or the same size as the analyte. An affinity reagent may form a reversible or irreversible bond with an analyte. An affinity reagent may bind with an analyte in a covalent or non-covalent manner. Affinity reagents may include reactive affinity reagents, catalytic affinity reagents (e.g., kinases, proteases, etc.) or non-reactive affinity reagents (e.g., antibodies or fragments thereof). An affinity reagent can be non-reactive and non-catalytic, thereby not permanently altering the chemical structure of an analyte to which it binds. Affinity reagents that can be particularly useful for binding to proteins include, but are not limited to, antibodies or functional fragments thereof (e.g., Fab′ fragments, F(ab′)₂ fragments, single-chain variable fragments (scFv), di-scFv, tri-scFv, or microantibodies), affibodies, affilins, affimers, affitins, alphabodies, anticalins, avimers, DARPins, monobodies, nanoCLAMPs, nucleic acid aptamers, protein aptamers, lectins or functional fragments thereof.

As used herein, the term “array” refers to a population of analytes (e.g. proteins) that are associated with unique identifiers such that the analytes can be distinguished from each other. A unique identifier can be, for example, a solid support (e.g. particle or bead), spatial address on a solid support, tag, label (e.g. luminophore), or barcode (e.g. nucleic acid barcode) that is associated with an analyte and that is distinct from other identifiers in the array. Analytes can be associated with unique identifiers by attachment, for example, via covalent bonds or non-covalent bonds (e.g. ionic bond, hydrogen bond, van der Waals forces, electrostatics etc.). An array can include different analytes that are each attached to different unique identifiers. An array can include different unique identifiers that are attached to the same or similar analytes. An array can include separate solid supports or separate addresses that each bear a different analyte, wherein the different analytes can be identified according to the locations of the solid supports or addresses.

As used herein, the term “attached” refers to the state of two things being joined, fastened, adhered, connected or bound to each other. Attachment can be covalent or non-covalent. For example, a particle can be attached to a protein by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions, adhesion, adsorption, and hydrophobic interactions.

As used herein, the term “binding affinity” or “affinity” refers to the strength or extent of binding between an affinity reagent and a binding partner. In some cases, the binding affinity of an affinity reagent for a binding partner may be vanishingly small or effectively zero. A binding affinity of an affinity reagent for a binding partner may be qualified as being a “high affinity,” “medium affinity,” or “low affinity.” A binding affinity of an affinity reagent for a binding partner, affinity target, or target moiety may be quantified as being “high affinity” if the interaction has a dissociation constant of less than about 100 nM, “medium affinity” if the interaction has a dissociation constant between about 100 nM and 1 mM, and “low affinity” if the interaction has a dissociation constant of greater than about 1 mM. Binding affinity can be described in terms known in the art of biochemistry such as equilibrium dissociation constant (K_{D}), equilibrium association constant (K_{A}), association rate constant (kₒₙ), dissociation rate constant (k_{off}) and the like. See, for example, Segel, Enzyme Kinetics John Wiley and Sons, New York (1975), which is incorporated herein by reference in its entirety.

As used herein, the term “binding probability” refers to the probability that an affinity reagent or probe may be observed to interact with an analyte, for example, within a given binding context. A binding probability may be expressed as a discrete number (e.g., 0.4 or 40%). A binding probability may include one or more factors, including binding specificity, likelihood of locating a target epitope, or the likelihood of binding for a sufficient time to detect a binding interaction.

The term “comprising” is intended herein to be open-ended, including not only the recited elements, but further encompassing any additional elements.

As used herein, the term “conformation,” when used in reference to a macromolecule or portion thereof, refers to the shape or proportionate dimensions of the macromolecule or portion. At the molecular level conformation can be characterized by the spatial arrangement of a macromolecule that results from the rotation of its atoms about their bonds. The conformational state of a macromolecule or portion thereof, such as a protein or nucleic acid, can be characterized in terms of secondary structure, tertiary structure, or quaternary structure. Secondary structure of a nucleic acid is the set of interactions between bases of the nucleic acid such as interactions formed by internal complementarity in a single stranded nucleic acid or by complementarity between two strands in a double helix. Tertiary structure of a nucleic acid is the three-dimensional shape of the nucleic acid as defined, for example, by the relative locations of its atoms in three-dimensional space. Quaternary structure of a nucleic acid is the overall shape resulting from interactions between two or more nucleic acids at a higher level than the secondary or tertiary levels. Secondary structure of a protein is the three-dimensional form of local segments of the protein which can be defined, for example, by the pattern of hydrogen bonds between the amino hydrogen and carboxyl oxygen atoms in the peptide backbone or by the regular pattern of backbone dihedral angles in a particular region of the Ramachandran plot for the protein. Tertiary structure of a protein is the three-dimensional shape of a single polypeptide chain backbone including, for example, interactions and bonds of side chains that form domains. Quaternary structure of a protein is the three-dimensional shape and interaction between the amino acids of multiple polypeptide chain backbones. A macromolecule having a given composition may be capable of adopting more than one conformational state with or without changes to its molecular composition. For example, a protein having a given amino acid sequence (i.e. protein primary structure) may adopt different conformations at the secondary, tertiary or quaternary level, and a nucleic acid having a given nucleotide sequence (i.e. nucleic acid primary structure) may adopt different conformations at the secondary, tertiary or quaternary level.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “epitope” refers to an affinity target within a protein, polypeptide or other analyte. An epitope may include a sequence of amino acids that are sequentially adjacent in the primary structure of a protein. An epitope may include amino acids that are structurally adjacent in the secondary, tertiary or quaternary structure of a protein despite being non-adjacent in the primary sequence of the protein. An epitope can include a moiety of a protein that arises due to a post-translational modification, such as a phosphate, phosphotyrosine, phosphoserine, phosphothreonine, or phosphohistidine. An epitope can optionally be recognized by or bound to an antibody. However, an epitope need not necessarily be recognized by any antibody, for example, instead being recognized by an aptamer, mini-protein or other affinity reagent. An epitope can optionally bind an antibody to elicit an immune response. However, an epitope need not necessarily participate in, nor be capable of, eliciting an immune response.

As used herein, the term “label” refers to a molecule or moiety that provides a detectable characteristic. The detectable characteristic can be, for example, an optical signal such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a fluorophore, luminophore, chromophore, nanoparticle (e.g., gold, silver, carbon nanotubes), heavy atoms, radioactive isotope, mass label, charge label, spin label, receptor, ligand, or the like. A label may produce a signal that is detectable in real-time (e.g., fluorescence, luminescence, radioactivity). A label may produce a signal that is detected off-line (e.g., a nucleic acid barcode) or in a time-resolved manner (e.g., time-resolved fluorescence). A label may produce a signal with a characteristic frequency, intensity, polarity, duration, wavelength, sequence, or fingerprint. A “labelling reagent” is a reagent that modifies a macromolecule or other object to include a label, for example, via attachment of a label moiety to the macromolecule or other object or via modification of a moiety of the macromolecule or other object to provide a detectable characteristic.

As used herein, the term “measurement outcome” refers to information resulting from observation, simulation or examination of a process. For example, the measurement outcome for contacting an affinity reagent with an analyte can be referred to as a “binding outcome.” A measurement outcome can be positive or negative. For example, observation of binding is a positive binding outcome and observation of non-binding is a negative binding outcome. A measurement outcome can be a null outcome in the event a positive or negative outcome is not apparent from a given measurement. A measurement outcome can be represented in binary terms, such as a zero (0) for a negative binding outcome and a one (1) for a positive binding outcome. In some cases a ternary representation can be used, for example, when zero (0) represents a negative binding outcome, one (1) represents a positive binding outcome, and two (2) represents a null outcome. It is also possible to use continuous or analog values, as opposed to integers or discrete values, to represent different measurement outcomes.

As used herein, the term “macromolecule” refers to a group of at least one thousand atoms held together by covalent bonds. A macromolecule may also include non-covalent bonds or atoms associated by non-covalent bonds so long as at least one thousand atoms of the macromolecule are connected via a continuous network of covalent bonds. Optionally, a macromolecule can be larger, for example, having at least 2.5×10³, 5×10³, 7.5×10³, 1×10⁴, 1×10⁵, 1×10⁶, or more atoms held together by covalent bonds. Exemplary macromolecules include, but are not limited to, linear polymers, branched polymers, naturally produced polymers, synthetic polymers, biologically active polymers and biologically inert polymers. Optionally macromolecules can be composed of proteins, nucleic acids, carbohydrates, lipids, or analogs thereof.

As used herein, the term “post translational modification” refers to a change to the chemical composition of a protein compared to the chemical composition encoded by the gene for the protein. Exemplary changes include those that alter the presence, absence or relative arrangement of different regions of amino acid sequence (e.g., splicing variants, or protein processing variants of a single gene), or due to presence or absence of different moieties on particular amino acids (e.g., post-translationally modified variants of a single gene). A post translational modification can be derived from an in vivo process or in vitro process. A post translational modification can be derived from a natural process or a synthetic process. Exemplary post translational modifications include those classified by the PSI-MOD ontology. See Smith, L. M. et al. Nat. Methods, 2013, 10, 186-187.

As used herein, the term “promiscuous,” when used in reference to a reagent, means that the reagent is known or suspected to react with a variety of different analytes in a given sample. For example, an affinity reagent that is known or suspected to recognize a variety of different analytes (e.g. a variety of proteins having different primary sequences) is promiscuous. A promiscuous reagent may be known or suspected of having high reactivity with one or more of the different analytes with which it reacts. For example, a promiscuous affinity reagent may have high affinity for one or more of the different analytes that it recognizes. A promiscuous reagent may be composed of a single type of reagent, such as a single affinity reagent, or a promiscuous reagent may be composed of two or more different types of reagents. For example, a promiscuous affinity reagent may be composed of a single type of antibody that recognizes a variety of different proteins in a sample, or the promiscuous affinity reagent may be composed of a pool containing several different antibody types that collectively recognize the variety of different proteins in the sample.

As used herein, the term “protein” refers to a molecule comprising two or more amino acids joined by a peptide bond. A protein may also be referred to as a polypeptide, oligopeptide or peptide. A protein can be a naturally-occurring molecule, or synthetic molecule. A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications. In some circumstances, different proteins may be distinguished from each other based on different genes from which they are expressed in an organism, different primary sequence length or different primary sequence composition. Proteins expressed from the same gene may nonetheless be different proteoforms, for example, being distinguished based on non-identical length, non-identical amino acid sequence or non-identical post-translational modifications. Different proteins can be distinguished based on one or both of gene of origin and proteoform state.

As used herein, the term “reaction extent” refers to the quantity of reactants in a reaction that react to form products of the reaction relative to the quantity of reactants in the reaction that do not react to form products. For example, the term can refer to the amount of first reactants (i.e. reactants of a first type) in a reaction that react with second reactants (i.e. reactants of a second type) to form products of the reaction relative to the amount of the first reactants in the reaction that do not react with the second reactants to form the products. Optionally, the products of the reaction can be covalently modified forms of one or more reactant types. For example, a reactant can be modified such that a moiety is attached to the reactant, a moiety is removed from the reactant, one or more moieties in the reactant are rearranged, a covalent bond is cleaved in the reactant, a covalent bond is formed in the reactant or a combination thereof. In some cases, the product of the reaction can be attachment of a first reactant to a second reactant or transfer of a moiety from a first reactant to a second reactant. In such cases, the first and second reactants can be the same or different types of reactants. Optionally, the products of the reaction are non-covalently modified forms of one or more reactant types. For example, a first reactant can be non-covalently bound to a second reactant. Accordingly, the term “binding extent” refers to the quantity of reactants in a binding reaction that bind to affinity reagents relative to the quantity of reactants in the binding reaction that do not bind to the affinity reagents. The binding extent can be determined, for example, at equilibrium or at a given pre-equilibrium timepoint.

As used herein, the term “single,” when used in reference to an object such as an analyte, means that the object is individually manipulated or distinguished from other objects. A single analyte can be a single molecule (e.g. single protein), a single complex of two or more molecules (e.g. a multimeric protein having two or more separable subunits, a single protein attached to a structured nucleic acid particle or a single protein attached to an affinity reagent), a single particle, or the like. Reference herein to a “single analyte” in the context of a composition, system or method herein does not necessarily exclude application of the composition, system or method to multiple single analytes that are manipulated or distinguished individually, unless indicated contextually or explicitly to the contrary.

As used herein, the term “single-analyte resolution” refers to the detection of, or ability to detect, an analyte on an individual basis, for example, as distinguished from its nearest neighbor in an array.

As used herein, the term “solid support” refers to a substrate that is insoluble in aqueous liquid. Optionally, the substrate can be rigid. The substrate can be non-porous or porous. The substrate can optionally be capable of taking up a liquid (e.g. due to porosity) but will typically, but not necessarily, be sufficiently rigid that the substrate does not swell substantially when taking up the liquid and does not contract substantially when the liquid is removed by drying.

A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In particular configurations, a flow cell contains the solid support such that fluids introduced to the flow cell can interact with a surface of the solid support to which one or more components of a binding event (or other reaction) is attached.

As used herein, the term “specificity,” when used in reference to an assay reagent, refers to the tendency of the assay reagent to preferentially react or interact with a given analyte relative to other analytes. For example, in the context of an affinity reagent, the term “specificity” refers to the tendency of the affinity reagent to preferentially bind with a given analyte relative to other analytes. An assay reagent may have a calculated, observed, known, or predicted specificity for a given analyte. Specificity may refer to selectivity for a single analyte in a sample relative to one, some or all other analytes in the sample. Moreover, specificity may refer to selectivity for a subset of analytes in a sample relative to at least one other analyte in the sample.

As used herein, the term “system” refers to a group of tangible components that are connected or work together to achieve a function. Optionally, a system can further include non-tangible components. However, a system need not include non-tangible components.

The embodiments set forth below and recited in the claims can be understood in view of the above definitions.

The present disclosure provides a method of determining accessibility of macromolecule structures. The method can include steps of (a) contacting an array of macromolecules with a plurality of different assay reagents, wherein individual addresses of the array are each attached to a single macromolecule, the macromolecule having a plurality of different reactive sites, and wherein the different assay reagents are different with respect to specificity for the reactive sites; (b) detecting reaction of the array of macromolecules with the different assay reagents, whereby the individual addresses of the array are resolved, and whereby the different assay reagents are resolved; (c) determining a first observed reaction extent comprising the fraction of the individual addresses having a first reactive site that is observed to react with a first assay reagent of the plurality of different assay reagents in step (b); (d) providing an expected reaction extent including the extent to which the assay reagent reacts with a candidate macromolecule having the same molecular structure as the macromolecules at the fraction of the individual addresses observed to react with the first assay reagent in step (b); and (e) determining accessibility of the first reactive site of the macromolecules based on a comparison of the observed reaction extent and the expected reaction extent.

Optionally, a method of determining accessibility of macromolecule structures can include the steps of (a) contacting an array of macromolecules with a plurality of different assay reagents, wherein individual addresses of the array are each attached to a single macromolecule, the macromolecule including a plurality of different reactive sites, and wherein the different assay reagents are different with respect to specificity for the reactive sites; (b) detecting reaction of the array of macromolecules with the different assay reagents, whereby the individual addresses of the array are resolved, and whereby the different assay reagents are resolved; (c) determining a first observed reaction extent including the fraction of the individual addresses observed to react with a first assay reagent in step (b); (d) determining a second observed reaction extent including the fraction of the individual addresses observed to react with a second assay reagent in step (b); (e) determining an observed double reaction extent including the fraction of the individual addresses observed to react with both the first assay reagent and the second assay reagent in step (b); (f) determining an expected double reaction extent from the first observed reaction extent and the second observed reaction extent; and (g) determining accessibility of a first reactive site of the plurality of different reactive sites based on a comparison of the observed double reaction extent and the expected double reaction extent.

The methods, compositions and systems of the present disclosure are particularly well suited for use with macromolecules. Macromolecules such as proteins can adopt any of a variety of conformations and these conformations can influence their function and/or the function of systems in which they are present. For example, protein function in biological systems is known to be exquisitely responsive to the conformational state of the protein, and changes in conformation of a protein are often correlated with, and sometimes causative of, changes in function of the protein. Methods, compositions and systems set forth herein can be useful in determining conformational states or chemically modified states of macromolecules such as proteins. This information can provide useful insights into the function of the macromolecules including, for example, how they are regulated in biological systems. This information can also be informative in determining the identity of a macromolecule of interest. For example, an extant macromolecule can be identified based on similarity of an observed conformation to known or suspected dynamics of a candidate macromolecule. Moreover, the information can be used as a basis for determining the quality of results from an assay, for example, based on the degree to which observed conformations are consistent, or inconsistent, with a range of conformations expected for one or more macromolecules being assayed.

Although macromolecules, such as proteins, are exemplified throughout the present disclosure, it will be understood that a method, composition or system of the present disclosure can be applied to other analytes, for example, to identify, characterize or quantify the analyte. Exemplary analytes include, but are not limited to, biomolecules, metabolites, hormones, vitamins, enzyme cofactors, therapeutic agents, candidate therapeutic agents, tissues, cells, organelles, subcellular particles, vesicles, viruses, or combinations thereof. An analyte can be a non-biological molecule, such as a synthetic polymer, metal, metal oxide, ceramic, semiconductor, mineral, or a combination thereof. Macromolecules other than proteins that can be used include, for example, carbohydrates (e.g. polysaccharides), nucleic acids (e.g. DNA or RNA), lipids or synthetic polymers.

One or more macromolecules or other analytes used herein, can be derived from a natural or synthetic source. Exemplary sources include, but are not limited to biological tissues, fluids, cells or subcellular compartments (e.g. organelles). For example, a sample can be derived from a tissue biopsy, biological fluid (e.g. blood, sweat, tears, plasma, extracellular fluid, urine, mucus, saliva, semen, vaginal fluid, synovial fluid, lymph, cerebrospinal fluid, peritoneal fluid, pleural fluid, amniotic fluid, intracellular fluid, extracellular fluid, etc.), fecal sample, hair sample, cultured cell, culture media, fixed tissue sample (e.g. fresh frozen or formalin-fixed paraffin-embedded) or product of a synthesis reaction. A source may include any sample where a macromolecule or other analyte of interest is a native or expected constituent. For example, a primary source for a cancer biomarker protein may be a tumor biopsy sample or bodily fluid. Other sources include environmental samples or forensic samples.

Exemplary organisms from which macromolecules or other analytes can be derived include, for example, a mammal such as a rodent, mouse, rat, rabbit, guinea pig, ungulate, horse, sheep, pig, goat, cow, cat, dog, primate, non-human primate or human; a plant such as Arabidopsis thaliana, tobacco, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a Dictyostelium discoideum; a fungi such as Pneumocystis carinii, Takifugu rubripes, yeast, Saccharamoyces cerevisiae or Schizosaccharomyces pombe; or a Plasmodium falciparum. Proteins can also be derived from a prokaryote such as a bacterium, Escherichia coli, staphylococci or Mycoplasma pneumoniae; an archae; a virus such as Hepatitis C virus, influenza virus, coronavirus, or human immunodeficiency virus; or a viroid. Macromolecules or other analytes of interest can be derived from a homogeneous culture or population of the above organisms or alternatively from a collection of several different organisms, for example, in a community or ecosystem.

In some cases, a macromolecule or other analyte can be derived from an organism that is collected from a host organism. For example, a macromolecule or other analyte may be derived from a parasitic, pathogenic, symbiotic, or latent organism collected from a host organism. A macromolecule or other analyte can be derived from an organism, tissue, cell or biological fluid that is known or suspected of being linked with a disease state or disorder (e.g., cancer). Alternatively, a macromolecule or other analyte can be derived from an organism, tissue, cell or biological fluid that is known or suspected of not being linked to a particular disease state or disorder. For example, a macromolecule or other analyte isolated from such a source can be used as a control for comparison to results acquired from a source that is known or suspected of being linked to the particular disease state or disorder. A sample may include a microbiome or substantial portion of a microbiome. In some cases, one or more analytes used in a method, composition or apparatus set forth herein may be obtained from a single source and no more than the single source. The single source can be, for example, a single organism (e.g. an individual human), single tissue, single cell, single organelle (e.g. endoplasmic reticulum, Golgi apparatus or nucleus), or single protein-containing particle (e.g., a viral particle or vesicle).

Particular configurations of the methods, compositions or systems set forth herein are applied to proteins. For example, a plurality of proteins having any of a variety of compositions, such as a plurality of proteins composed of a proteome or fraction thereof, can be used. Optionally, a plurality of proteins can include solution-phase proteins, such as proteins in a biological sample or fraction thereof, or a plurality of proteins can include proteins that are immobilized, such as proteins attached to addresses of an array or other solid support. By way of further example, a plurality of proteins can include proteins that are detected, analyzed or identified in connection with a method, composition or apparatus of the present disclosure. The content of a plurality of proteins can be understood according to any of a variety of characteristics such as those set forth below or elsewhere herein.

A plurality of proteins can be characterized in terms of total protein mass. The total mass of protein in a liter of plasma has been estimated to be 70 g and the total mass of protein in a human cell has been estimated to be between 100 pg and 500 pg depending upon cells type. See Wisniewski et al. Molecular & Cellular Proteomics 13:10.1074/mcp.M113.037309, 3497-3506 (2014), which is incorporated herein by reference. A plurality of proteins used or included in a method, composition or system set forth herein can include at least 1 pg, 10 pg, 100 pg, 1 ng, 10 ng, 100 ng, 1 μg, 10 μg, 100 μg, 1 mg, 10 mg, 100 mg or more protein by mass. Alternatively or additionally, a plurality of proteins may contain at most 100 mg, 10 mg, 1 mg, 100 μg, 10 μg, 1 μg, 100 ng, 10 ng, 1 ng, 100 pg, 10 pg, 1 pg or less protein by mass.

A plurality of proteins can be characterized in terms of percent mass relative to a given source such as a biological source (e.g. cell, tissue, or biological fluid such as blood). For example, a plurality of proteins may contain at least 60%, 75%, 90%, 95%, 99%, 99.9% or more of the total protein mass present in the source from which the plurality of proteins was derived. Alternatively or additionally, a plurality of proteins may contain at most 99.9%, 99%, 95%, 90%, 75%, 60% or less of the total protein mass present in the source from which the plurality of proteins was derived.

A plurality of proteins can be characterized in terms of total number of protein molecules. The total number of protein molecules in a Saccharomyces cerevisiae cell has been estimated to be about 42 million protein molecules. See Ho et al., Cell Systems (2018), DOI: 10.1016/j.cels.2017.12.004, which is incorporated herein by reference. A plurality of proteins used or included in a method, composition or system set forth herein can include at least 2 protein molecules, 10 protein molecules, 100 protein molecules, 1×10⁴ protein molecules, 1×10⁶ protein molecules, 1×10⁸ protein molecules, 1×10¹⁰ protein molecules, 1 mole (6.02214076×10²³ molecules) of protein, 10 moles of protein molecules, 100 moles of protein molecules or more. Alternatively or additionally, a plurality of proteins may contain at most 100 moles of protein molecules, 10 moles of protein molecules, 1 mole of protein molecules, 1×10¹⁰ protein molecules, 1×10⁸ protein molecules, 1×10⁶ protein molecules, 1×10⁴ protein molecules, 100 protein molecules, 10 protein molecules, or 2 protein molecules.

A plurality of proteins can be characterized in terms of the variety of full-length primary protein structures in the plurality. For example, the variety of full-length primary protein structures in a plurality of proteins can be equated with the number of different protein-encoding genes in the source for the plurality of proteins. Whether or not the proteins are derived from a known genome or from any genome at all, the variety of full-length primary protein structures can be counted independent of presence or absence of post translational modifications in the proteins. A human proteome is estimated to have about 20,000 different protein-encoding genes such that a plurality of proteins derived from a human can include up to about 20,000 different primary protein structures. See Aebersold et al., Nat. Chem. Biol. 14:206-214 (2018), which is incorporated herein by reference. Other genomes and proteomes in nature are known to be larger or smaller. A plurality of proteins used or included in a method, composition or system set forth herein can have a complexity of at least 2, 5, 10, 100, 1×10³, 1×10⁴, 2×10⁴, 3×10⁴ or more different full-length primary protein structures. Alternatively or additionally, a plurality of proteins can have a complexity that is at most 3×10⁴, 2×10⁴, 1×10⁴, 1×10³, 100, 10, 5, 2 or fewer different full-length primary protein structures.

In relative terms, a plurality of proteins used or included in a method, composition or system set forth herein may contain at least one representative for at least 60%, 75%, 90%, 95%, 99%, 99.9% or more of the proteins encoded by the genome of the source from which the plurality was derived. Alternatively or additionally, a plurality of proteins may contain a representative for at most 99.9%, 99%, 95%, 90%, 75%, 60% or less of the proteins encoded by the genome of the source from which the plurality was derived.

A plurality of proteins can be characterized in terms of the variety of primary protein structures in the plurality including transcribed splice variants. The human proteome has been estimated to include about 70,000 different primary protein structures when splice variants ae included. See Aebersold et al., Nat. Chem. Biol. 14:206-214 (2018), which is incorporated herein by reference. Moreover, the number of the partial-length primary protein structures can increase due to fragmentation that occurs in a sample. A plurality of proteins used or included in a method, composition or system set forth herein can have a complexity of at least 2, 5, 10, 100, 1×10³, 1×10⁴, 1×10⁵, 1×10⁶, 1×10⁸, 1×10¹⁰, or more different primary protein structures. Alternatively or additionally, a plurality of proteins can have a complexity that is at most 1×10¹⁰, 1×10⁸, 1×10⁶, 1×10⁵, 5×10⁴, 1×10⁴, 1×10³, 100, 10, 5, 2 or fewer different primary protein structures.

A plurality of proteins can be characterized in terms of the variety of protein structures in the plurality including different primary structures and/or different proteoforms among the primary structures. Different molecular forms of proteins expressed from a given gene are considered to be different proteoforms. Proteoforms can differ, for example, due to differences in primary structure (e.g. shorter or longer amino acid sequences), different arrangement of domains (e.g. transcriptional splice variants), or different post translational modifications (e.g. presence or absence of phosphoryl, glycosyl, acetyl, or ubiquitin moieties). The human proteome is estimated to include hundreds of thousands of proteins when counting the different primary structures and proteoforms. See Aebersold et al., Nat. Chem. Biol. 14:206-214 (2018), which is incorporated herein by reference. A plurality of proteins used or included in a method, composition or system set forth herein can have a complexity of at least 2, 5, 10, 100, 1×10³, 1×10⁴, 1×10⁵, 1×10⁶, 5×10⁶, 1×10⁷ or more different protein structures. Alternatively or additionally, a plurality of proteins can have a complexity that is at most 1×10⁷, 5×10⁶, 1×10⁶, 1×10⁵, 1×10⁴, 1×10³, 100, 10, 5, 2 or fewer different protein structures.

A plurality of proteins can be characterized in terms of the dynamic range for the abundance of different protein structures in the sample. The dynamic range can be a measure of the range of abundance for all different protein structures in a plurality of proteins, the range of abundance for all different primary protein structures in a plurality of proteins, the range of abundance for all different full-length primary protein structures in a plurality of proteins, the range of abundance for all different full-length gene products in a plurality of proteins, the range of abundance for all different proteoforms expressed from a given gene, or the range of abundance for any other set of different proteins set forth herein. The dynamic range for all proteins in human plasma is estimated to span more than 10 orders of magnitude from albumin, the most abundant protein, to the rarest proteins that have been measured clinically. See Anderson and Anderson Mol Cell Proteomics 1:845-67 (2002), which is incorporated herein by reference. The dynamic range for plurality of proteins set forth herein can be a factor of at least 10, 100, 1×10³, 1×10⁴, 1×10⁶, 1×10⁸, 1×10¹⁰, or more. Alternatively or additionally, the dynamic range for plurality of proteins set forth herein can be a factor of at most 1×10¹⁰, 1×10⁸, 1×10⁶, 1×10⁴, 1×10³, 100, 10 or less.

Some configurations of the compositions, systems or methods set forth herein, can employ different proteoforms, such as proteins having the same primary structure (i.e. the same sequence of amino acids) but differing with respect to the number, type, or location of post-translational modifications. Methods of the present disclosure can be configured to identify a number, type, or location for one or more post-translational modifications in one or more proteins of a sample. The conformational state of one or more proteoforms can also be determined or characterized. Exemplary post-translational modifications include, but are not limited to, myristoylation, palmitoylation, isoprenylation, prenylation, farnesylation, geranylgeranylation, lipoylation, flavin moiety attachment, Heme C attachment, phosphopantetheinylation, retinylidene Schiff base formation, dipthamide formation, ethanolamine phosphoglycerol attachment, hypusine, beta-Lysine addition, acylation, acetylation, deacetylation, formylation, alkylation, methylation, C-terminal amidation, arginylation, polyglutamylation, polyglyclyation, butyrylation, gamma-carboxylation, glycosylation, glycation, polysialylation, malonylation, hydroxylation, iodination, nucleotide addition, phosphoate ester formation, phosphoramidate formation, phosphorylation, adenylylation, uridylylation, propionylation, pyrolglutamate formation, S-glutathionylation, S-nitrosylation, S-sulfenylation, S-sulfinylation, S-sulfonylation, succinylation, sulfation, glycation, carbamylation, carbonylation, isopeptide bond formation, biotinylation, carbamylation, oxidation, reduction, pegylation, ISGylation, SUMOylation, ubiquitination, neddylation, pupylation, citrullination, deamidation, elminylation, disulfide bridge formation, isoaspartate formation, and racemization.

A post-translational modification may occur at a particular type of amino acid residue in a protein. For example, the phosphate moiety of a particular proteoform can be present on a serine, threonine, tyrosine, histidine, cysteine, lysine, aspartate or glutamate residue. In another example, an acetyl moiety of a particular proteoform can be present on the N-terminus or on a lysine of a protein. In another example, a serine or threonine residue of a proteoform can have an O-linked glycosyl moiety, or an asparagine residue of a proteoform can have an N-linked glycosyl moiety. In another example, a proline, lysine, asparagine, aspartate or histidine amino acid of a proteoform can be hydroxylated. In another example, a proteoform can be methylated at an arginine or lysine amino acid. In another example, a proteoform can be ubiquitinated at the N-terminal methionine or at a lysine amino acid. Some amino acids are known to be inert to particular post-translational modifications, such modifications resulting in addition of moieties to their sidechains. Examples of such amino acids include, but are not limited to, alanine, valine, isoleucine, leucine, and phenylalanine.

Nucleic acids are a type of macromolecule that can be used in a method, composition or system set forth herein. Particularly useful nucleic acids include, but are not limited to, deoxyribonucleic acid (DNA), ribonucleic acid (RNA), peptide nucleic acid (PNA) or analogs thereof. Nucleic acids can be partially or fully single stranded. Nucleic acids can be partially or fully double stranded. Another type of macromolecule that can be used in a method, composition or system set forth herein is a carbohydrate. Particularly useful carbohydrates include, but are not limited to, oligosaccharides or polysaccharides. The oligosaccharides can be linear oligosaccharides or branched oligosaccharides, and the polysaccharides can be linear polysaccharides or branched polysaccharides. Synthetic polymers are another type of macromolecule that can be used in a method, composition or system set forth herein. Examples include, but are not limited to, plastics and fibers.

A macromolecule that is used in a method, composition or system of the present disclosure may be capable of adopting one or more conformational states. Optionally, a macromolecule can be in a native conformational state. For example, macromolecule can be in a native conformational state when attached to a solid support (e.g. at an address of an array), when contacted with an assay reagent, or when being detected. In the case of a biologically derived macromolecule, or analog thereof, a native conformational state can be a conformational state adopted in a native milieu such as within a biological fluid, tissue, cell, cytosol, membrane or organelle. Taking proteins as an example, native conformational state can be associated with presence or absence of a particular post translational modifications, such as presence or absence of a post-translational moiety; presence or absence of a protein domain, such as a signal sequence, pre-sequence, pro-sequence, amino-terminal domain, carboxy-terminal domain, or internal domain that is removed during protein processing; or presence or absence of a disulfide bond.

In some configurations of a method, composition or system set forth herein, a macromolecule can adopt a denatured conformation. A denatured conformation of a macromolecule can be characterized as the macromolecule having greater degrees of conformation freedom compared to a native state. For example, denatured proteins can be in a molten globule state. A denatured conformation of a macromolecule can be characterized as the macromolecule lacking one or more activity of the native conformation. For example, a denatured protein may lack one or more enzymatic activity of the native protein. Optionally, a macromolecule can be in a denatured conformational state when attached to a solid support (e.g. at an address of an array), when contacted with an assay reagent, or when being detected. Denaturation can result in change of secondary structure, tertiary structure or quaternary structure, compared to the structure of the macromolecule in a native conformation. Denaturation need not result in changes to the primary structure of a macromolecule.

Macromolecule can be denatured by heat, agitation, radiation, electrical current, pH, salt concentration, chemical denaturants, or other known conditions. Macromolecules can be denatured at temperatures that exceed temperatures of the biological system from which the proteins are derived. For example, macromolecules can be denatured at temperatures above 37° C., 40° C., 50° C., 60° C., 70° C., 80° C., 90° C., or higher. In some cases, for example, when using proteins, heat can be applied to denature the proteins and the proteins can remain in a denatured state after return to a lower temperature. Alternatively, heat can be maintained throughout one or more steps set forth herein such that the protein or other macromolecule remains in a denatured state. Macromolecules can be denatured at pH outside of the range for the biological system from which the macromolecules are derived. For example, pH below 7.0, 6.0, 5.0, 4.0, 3.0, 2.0 or 1.0 can be used. Alternatively, pH higher than 7.0, 8.0, 9.0, 10.0, 11.0, 12.0, 13.0 or 14.0 can be used. In some cases, for example, when using proteins, extreme pH can be applied to denature the proteins and the proteins can remain in a denatured state after return to a lower or higher pH. Alternatively, extreme pH can be maintained throughout one or more steps set forth herein such that the protein, or other macromolecule, remains in a denatured state. Macromolecules, such as proteins, can be denatured at by chemical denaturants including, for example, detergents, such as sodium dodecyl sulfate; alcohols, such as ethanol; organic solvents, such as chloroform, dimethylsulfoxide, formamide, propylene glycol or acetonitrile; chaotropic agents, such as urea, guanidinium chloride, or lithium perchlorate; or reducing agents such as 2-mercaptoethanol, dithiothreitol, or tris(2-carboxyethyl)phosphine.

A method of the present disclosure can optionally include a step of denaturing one or more macromolecules, for example, by applying a denaturant set forth herein. A macromolecule can be denatured prior to being extracted from its native milieu, prior to being separated from other components of its native milieu, prior to being isolated from its native milieu, prior to being attached to a solid support, prior to being contacted with one or more assay reagents, or prior to being detected. Alternatively or additionally, denaturation of a macromolecule can occur after one or more step of a method set forth herein, for example, after extracting the macromolecule from its native milieu, after separating the macromolecule from other components of its native milieu, after isolating the macromolecule from its native milieu, after attaching the macromolecule to a solid support, after contacting the macromolecule with one or more assay reagents, or after detecting the macromolecule. Denaturation can be carried out between steps of a method set forth herein. For example, a macromolecule can be assayed while in a native state and then the macromolecule can be denatured and assayed in a denatured state. Comparison of assay results for a macromolecule in native and denatured conformations can provide information regarding the identity, quantity, structural characteristics or functional characteristics of the macromolecule.

A method of the present disclosure can optionally include a step of folding one or more macromolecules, for example, by removing a denaturant set forth herein. Folding can, in some cases, return one or more macromolecule to a native conformation. Alternatively, folding can cause the macromolecule to adopt an alternative conformation that although not a native conformation is nevertheless more conformationally constrained than the denatured conformation. A macromolecule can be subjected to folding prior to being extracted from its native milieu, prior to being separated from other components of its native milieu, prior to being isolated from its native milieu, prior to being attached to a solid support, prior to being contacted with one or more assay reagents, or prior to being detected. Alternatively or additionally, folding can occur after one or more step of a method set forth herein, for example, after the macromolecule is extracted from its native milieu, after the macromolecule is separated from other components of its native milieu, after the macromolecule is isolated from its native milieu, after the macromolecule is attached to a solid support, after the macromolecule is contacted with one or more assay reagents, or after the macromolecule is detected. Folding can be carried out between steps of a method set forth herein. For example, a macromolecule can be assayed while in a denatured conformation and then the macromolecule can be folded and assayed in the folded state. Comparison of assay results for a macromolecule in denatured and subsequently folded conformations can provide information regarding the identity, quantity, structural characteristics or functional characteristics of the macromolecule.

A method set forth herein can be carried out in a fluid phase or on a solid phase. For fluid phase configurations, a fluid containing one or more macromolecules can be mixed with another fluid containing one or more assay reagents. For solid phase configurations one or more macromolecules or assay reagents can be attached to a solid support. One or more components that will participate in a method set forth herein can be contained in a fluid and the fluid can be delivered to the solid support, the solid support being attached to one or more other component that will participate in the method. For example, a method of the present disclosure can include a step of contacting an array of macromolecules with a plurality of different assay reagents. Optionally, the macromolecule at each address of the array includes a plurality of different reactive sites, and the different assay reagents are different with respect to specificity for the reactive sites.

A method of the present disclosure can employ detection at single analyte resolution. A single analyte (e.g. a single protein or other macromolecule) may be resolved from other analytes based on, for example, spatial or temporal separation from the other analytes. For example, a method of the present disclosure can include a step of contacting an array of macromolecules with a plurality of different assay reagents. Optionally, the individual addresses of the array are each attached to a single macromolecule. As a further option, the method can include a step of detecting reaction of the array of macromolecules with a plurality of different assay reagents, whereby the individual addresses of the array are resolved from each other, and whereby assay reagents of different types are resolved from each other.

An alternative to single-analyte resolution is ensemble-level resolution or bulk-level resolution. Bulk-level resolution configurations can acquire a composite signal from a plurality of different analytes or affinity reagents in a vessel or on a surface. For example, a composite signal can be acquired from a population of different protein-affinity reagent complexes in a well or cuvette, or on a solid support surface, such that individual complexes are not resolved from each other. Ensemble-level resolution configurations can acquire a composite signal from a first ensemble of analytes or assay reagents in a sample, such that the composite signal is distinguishable from signals generated by a second ensemble of analytes or assay reagents in the sample. For example, the ensembles can be located at different addresses in an array. Accordingly, the composite signal obtained from each address will be an average of signals from the ensemble, yet signals from different addresses can be distinguished from each other.

Whether configured for single-analyte resolution, bulk-level resolution or ensemble-level resolution, a composition, system or method set forth herein can be configured to contact a plurality of different macromolecules (e.g. an array of different macromolecules) with a plurality of different assay reagents. For example, a plurality of assay reagents (whether configured separately or as a pool) may include at least 2, 5, 10, 25, 50, 100, 250, 500, 1000 or more types of assay reagents, each type of assay reagent differing from the other types with respect to the reactive site recognized. Alternatively or additionally, a plurality of assay reagents may include at most 1000, 500, 250, 100, 50, 25, 10, 5, or 2 types of assay reagents, each type of assay reagent differing from the other types with respect to the reactive site recognized. Different types of assay reagents in a pool can be uniquely labeled such that the different types can be distinguished from each other. In some configurations, at least two, and up to all, of the different types of assay reagents in a pool may be indistinguishably labeled. Alternatively or additionally to the use of unique labels, different types of assay reagents can be delivered and detected serially when evaluating one or more macromolecules (e.g. in an array). Although several aspects of the methods of the present disclosure are exemplified for multiplex configurations, it will be understood that the methods and steps therein can be applied to detection and characterization of one macromolecule, the one macromolecule being presented at single-molecule resolution, in bulk, or in an ensemble of macromolecules.

In multiplexed formats, different macromolecules that are to be detected can be attached to different unique identifiers (e.g. addresses forming an array on a solid support), and the macromolecules can be manipulated and detected in parallel. For example, a fluid containing one or more different assay reagents can be delivered to an array such that the macromolecules of the array are in simultaneous contact with the assay reagent(s). Moreover, a plurality of addresses can be observed in parallel allowing for rapid detection of assay outcomes (e.g. binding outcomes). A plurality of different macromolecules can have a complexity of at least 5, 10, 100, 1×10³, 1×10⁴, 2×10⁴, 3×10⁴ or more different macromolecules (e.g. each macromolecule being a native-length protein primary sequences). Alternatively or additionally, a plurality of different macromolecules can have a complexity of at most 3×10⁴, 2×10⁴, 1×10⁴, 1×10³, 100, 10, 5 or fewer different macromolecules (e.g. each macromolecule being a native-length protein primary sequences).

An array can be configured to separate addresses on a solid support. For example, addresses can be separated by less than 100 microns, 10 microns, 1 micron, 500 nm, 100 nm, 10 nm or less. Alternatively or additionally, an array can have addresses that are separated by at least 10 nm, 100 nm, 500 nm, 1 micron, 5 microns, 10 microns, 50 microns, 100 microns or more. Addresses in an array can each occupy an area on a solid support of less than 1 square millimeter, 500 square microns, 100 square microns, 25 square microns, 1 square micron or less. Alternatively or additionally, addresses can each have an area of more than 1 square micron, 25 square microns, 100 square microns, 500 square microns, 1 square millimeter or more.

An array can include at least about 1×10⁴, 1×10⁵, 1×10⁶, 1×10⁸, 1×10¹⁰, 1×10¹², or more unique identifiers (e.g. addresses on a solid support). Alternatively or additionally, an array can include at most 1×10¹², 1×10¹⁰, 1×10⁸, 1×10⁶, 1×10⁵, 1×10⁴ or fewer unique identifiers. At least one, some or all addresses of an array can be occupied by a macromolecule or other analyte of interest. For example, at least 50%, 75%, 90%, 95%, 99% or more of the addresses in an array can be occupied. It will be understood that the number of unique identifiers of an array that are attached to a macromolecule can differ from the number of different macromolecule types in the array. For example, an array can include a plurality of macromolecules of a given composition or type. When the macromolecules are proteins, the plurality of proteins in an array can constitute a proteome or subfraction of a proteome. Exemplary compositions of proteomes that can be present in an array include, but are not limited to, those set forth previously herein. The total number of proteins of a sample that is detected, characterized or identified can differ from the number of different primary sequences in the sample, for example, due to the presence of multiple copies of at least some protein types. Moreover, the total number of proteins of a sample that is detected, characterized or identified can differ from the number of candidate proteins suspected of being in the sample, for example, due to the presence of multiple copies of at least some protein types, absence of some proteins in a source for the sample, presence of unexpected proteins in a source for the sample, or loss of some proteins prior to analysis.

A macromolecule (e.g. protein) can be attached to a unique identifier (e.g. address of an array) using any of a variety of means. The attachment can be covalent or non-covalent. Exemplary covalent attachments include chemical linkers such as those produced using click chemistry or other linkages known in the art or described in US Pat. App. Pub. No. 2021/0101930 A1, which is incorporated herein by reference. Non-covalent attachment can be mediated by receptor-ligand interactions (e.g. (strept)avidin-biotin, antibody-antigen, or complementary nucleic acid strands), for example, wherein the receptor is attached to the unique identifier and the ligand is attached to the macromolecule, or vice versa. In particular configurations, a macromolecule (e.g. protein) is attached to a solid support (e.g. at an address in an array) via a structured nucleic acid particle (SNAP). A macromolecule can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful. SNAPs and other moieties can be used to attach macromolecules to unique identifiers such as tags or addresses in an array, for example, as set forth in US Pat. App. Pub. No. 2021/0101930 A1, WO 2021/087402 A1, or U.S. patent application Ser. No. 17/692,035 (issued as U.S. Pat. No. 11,505,796), each of which is incorporated herein by reference.

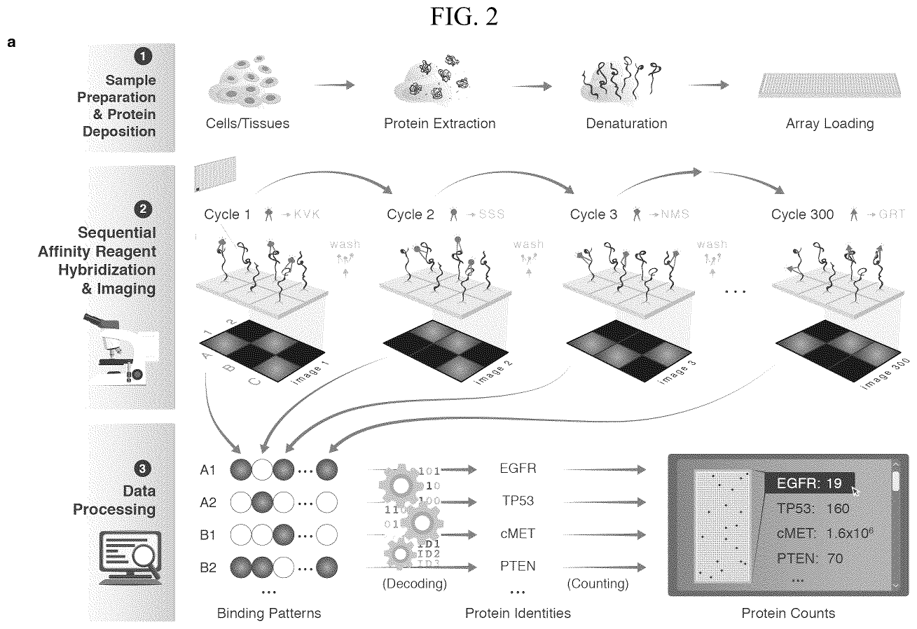

Useful techniques and steps for use in a method of the present disclosure can be understood in the context of assays configured to detect or characterize proteins. Those skilled in the art will recognize that such techniques and steps can be modified for use with other macromolecules or analytes of interest. An exemplary assay format is shown diagrammatically in FIG. 2. Proteins can be extracted from a sample and attached to an array. The array can be configured to have a plurality of addresses, wherein individual addresses are each attached to an individual protein, respectively, from the sample. The proteins that are attached to the array can be in a denatured state or native state. Optionally, a structured nucleic acid particle (SNAP) can mediate attachment of each protein to its respective address. Other linkers or attachment chemistry that can be used additionally or alternatively to SNAPs include, but are not limited to, those set forth in US Pat. App. Pub. No. 2021/0101930 A1, WO 2021/087402 A1, or U.S. Pat. App. Ser. No. 63/159,500 (and U.S. Pat. No. 11,505,796, which claims priority thereto), each of which is incorporated herein by reference.

Typically, the identity of the protein at any given address is not known (as such, the proteins may be referred to as ‘unknown’ proteins). Methods set forth herein can be used to identify proteins at one or more addresses in the array. Accordingly, the methods can be used to locate extant proteins in an array. A reaction can be carried out between arrayed proteins and a plurality of different assay reagents (e.g. affinity reagents). The number of addresses that react with a given assay reagent can be counted and, optionally, the number of addresses that do not react with a given assay reagent can be counted. The quantification results can be used to determine the reaction extent (e.g. binding extent). Continuing with the example diagrammed in FIG. 2, a plurality of affinity reagents (e.g. antibodies, aptamers, or functional fragments thereof), tagged with fluorophores, can be contacted with the array, and fluorescence can be detected from individual addresses to determine binding outcomes. The affinity reagents can be delivered to the array and detected serially as shown, such that each cycle detects binding outcomes for an individual affinity reagent. A first affinity reagent can be removed prior to delivering a second affinity reagent. In some configurations of the methods set forth herein, a plurality of different affinity reagents can be delivered in a cycle. The different affinity reagents that are delivered in a given cycle can be configured as a pool of indistinguishably labeled reagents (or they can lack labels), such that the different reagents are not distinguished in the detection step. Alternatively, two or more different affinity reagents that are delivered in a given cycle can be distinguishably labeled. As such, the affinity reagents can be distinguishably detected when bound to proteins on the array. The use of fluorescent labels and fluorescent detection is exemplary. Other labels and other detectors can be used such as those set forth herein or known in the art.

Further examples of reagents and techniques that can be used to detect proteins or other analytes in a method, system or composition of the present disclosure are set forth, for example, in U.S. Pat. No. 10,473,654 or US Pat. App. Pub. Nos. 2020/0318101 A1 or 2020/0286584 A1; U.S. Pat. App. Ser. No. 63/254,420, US Pat. App. Pub. No. 2023/0114905 A1, or Egertson et al., BioRxiv (2021), DOI: 10.1101/2021.10.11.463967, each of which is incorporated herein by reference. Exemplary methods, systems and compositions are set forth in further detail below.

Any of a variety of affinity reagents can be used in a composition, system or method set forth herein. An affinity reagent can be characterized, for example, prior to use in a method set forth herein, with respect to its binding properties. Exemplary binding properties include, but are not limited to, specificity, strength of binding; equilibrium binding constant (e.g. K_{A} or K_{D}); binding rate constant, such as association rate constant (kₒₙ) or dissociation rate constant (k_{off}); binding probability; or the like. Binding properties can be determined with regard to an epitope, a set of epitopes (e.g. a set of proteins having structural similarities), a macromolecule (e.g. protein), a set of macromolecules (e.g. a set of proteins having structural similarities), or a proteome.

An assay reagent can selectively react with one or more reactive sites compared to other reactive sites. For example, a reactive site can be an epitope. An affinity reagent can recognize, or bind, an epitope containing a particular group of amino acids in a protein. The group can occur as a contiguous sequence of amino acids in the protein or as a spatial grouping in which the amino acids are not necessarily contiguous in the linear sequence of the protein. An affinity reagent can be selective for one or more groups of amino acids compared to other groups of amino acids that are present in a protein or population of proteins. In some cases, an affinity reagent can have differential affinity for a group of amino acids depending on the presence or absence of a post-translational modification to the group. For example, an affinity reagent can recognize or bind a given group of amino acids that is post-translationally modified without substantially recognizing or binding the group of amino acids lacking the modification. Alternatively, an affinity reagent can recognize or bind a given group of amino acids that lacks one or more post-translational modification without substantially recognizing or binding the group of amino acids when the one or more post-translational modification is present.

An affinity reagent can include a label. Exemplary labels include, without limitation, a fluorophore, luminophore, chromophore, nanoparticle (e.g., gold, silver, carbon nanotubes), heavy atom, radioactive isotope, mass label, charge label, spin label, receptor, ligand, nucleic acid barcode, polypeptide barcode, polysaccharide barcode, or the like. A label can produce any of a variety of detectable signals including, for example, an optical signal such as absorbance of radiation, luminescence (e.g. fluorescence or phosphorescence) emission, luminescence lifetime, luminescence polarization, or the like; Rayleigh and/or Mie scattering; magnetic properties; electrical properties; charge; mass; radioactivity or the like. A label may produce a signal with a characteristic frequency, intensity, polarity, duration, wavelength, sequence, or fingerprint. A label need not directly produce a signal. For example, a label can bind to a receptor or ligand having a moiety that produces a characteristic signal. Such labels can include, for example, nucleic acids that are encoded with a particular nucleotide sequence, avidin, biotin, non-peptide ligands of known receptors, or the like.

A method of the present disclosure can include a step of reacting a macromolecule with an assay reagent to determine a measurement outcome. For example, the measurement outcome for contacting an affinity reagent with a macromolecule, such as a protein, can be observed as a binding outcome. A measurement outcome can be positive or negative. For example, observation of binding is a positive binding outcome and observation of non-binding is a negative binding outcome. A measurement outcome can be a null outcome, for example, when a positive binding outcome cannot be distinguished from a negative binding outcome. A plurality of measurement outcomes for a given macromolecule, for example, a macromolecule present at a particular address or other unique identifier in an array, can be combined into an empirical outcome profile. A decoding method can be used to identify or characterize macromolecules based on the empirical outcome profile and one or more candidate outcome profiles. A candidate outcome profile can be provided for each of one or more macromolecules that are suspected of being present in the array from which the empirical outcome profile is obtained. The candidate outcome profile for a given candidate macromolecule will typically include expected results for reaction of the candidate macromolecule with each of the assay reagents to which the array is subjected. For example, the expected results can be expressed as a probability that each assay reagent will react with the macromolecule. A plurality of candidate outcome profiles, for example in the form of a matrix of candidate outcome profiles, can be compared to an empirical outcome profile for a given macromolecule (e.g. a macromolecule at a given unique identifier in an array) and the macromolecule can be identified as the candidate macromolecule having an outcome profile that is most compatible with the empirical outcome profile.

Accordingly, one or more macromolecules can be identified by (1) performing reactions using assay reagents that react with candidate macromolecules suspected of being present in a given sample, wherein the known or predicted results of the reaction of each assay reagent with each of the candidate macromolecules is provided as a candidate measurement outcome, and wherein the candidate measurement outcomes for a given candidate macromolecule with a plurality of the assay reagents are combined into a candidate outcome profile, (2) subjecting one or more extant macromolecules to the plurality of assay reagents and detecting reaction results, wherein the result of reaction for each assay reagent with each of the extant macromolecules produces an empirical measurement outcome, and wherein the empirical measurement outcomes for a given extant macromolecule with the plurality of the assay reagents produces an empirical outcome profile, and (3) performing a decoding method that compares the empirical outcome profile for each extant macromolecule to the candidate outcome profiles using a machine learning model and identifying the extant macromolecule as the candidate macromolecule having the candidate outcome profile that is most compatible with its empirical outcome profile. The decoding methods will be exemplified below in the context of binding reactions. It will be understood that the decoding methods can be extended to other reactions beyond binding reactions.

A measurement outcome can be acquired using any of a variety of detection techniques that are appropriate to the reaction components used. For example, binding can be detected by acquiring a signal from a label attached to an affinity reagent when the affinity reagent is bound to an observed macromolecule, acquiring a signal from a label attached to a macromolecule when the macromolecule is bound to an observed affinity reagent, or acquiring signal(s) from labels attached to an affinity reagent and macromolecule when bound to each other. In some configurations a complex between a macromolecule and affinity reagent need not be directly detected, for example, in formats where a nucleic acid tag or other moiety is created or modified due to binding between the macromolecule and affinity reagent. Optical detection techniques such as luminescence intensity detection, luminescence lifetime detection, luminescence polarization detection, or surface plasmon resonance detection can be useful. Other detection techniques include, but are not limited to, electronic detection such as techniques that utilize a field-effect transistor (FET), ion-sensitive FET, or chemically-sensitive FET. Exemplary methods are set forth in U.S. Pat. No. 10,473,654 or U.S. patent application Ser. No. 17/523,869 (issued as U.S. Pat. No. 11,692,217), each of which is incorporated herein by reference.

A binding assay can be configured to identify a number of different macromolecules that exceeds the number of affinity reagents used. Taking proteins as exemplary macromolecules, the number of proteins identified can be at least 5×, 10×, 25×, 50×, 100× or more than the number of affinity reagents used. One or more proteins can be identified by (1) performing binding reactions using promiscuous affinity reagents that bind to multiple different candidate proteins suspected of being present in a given sample, (2) subjecting one or more proteins to a set of the promiscuous affinity reagents that, taken as a whole, produce an empirical binding profile for each protein, and (3) performing a decoding method that evaluates the empirical binding profile according to a binding model for binding of the promiscuous binding reagents to a plurality of candidate proteins, thereby identifying each of the one or more of the proteins based on compatibility with a respective candidate protein.

Promiscuity of an affinity reagent is a characteristic that can be understood relative to a given population of macromolecules. Continuing with proteins as exemplary macromolecules, promiscuity can arise due to the affinity reagent recognizing an epitope that is present in a plurality of different proteins that are known or suspected of being in a sample, such as a human proteome sample. For example, a promiscuous affinity reagent may recognize epitopes having relatively short amino acid lengths such as dimers, trimers, tetramers, pentamers or hexamers, wherein the epitopes are expected to occur in a substantial number of different proteins in a proteome of a human or other species. Alternatively or additionally, a promiscuous affinity reagent can recognize different epitopes (i.e. epitopes having a variety of different structures), the different epitopes being present in a plurality of different proteins in a proteome sample. For example, a promiscuous affinity reagent can have a high probability of binding to a primary epitope target and lesser probability for binding to one or more secondary epitope targets, the secondary epitope targets having a different sequence of amino acids when compared to the primary epitope target. Optionally, the secondary epitope targets can be biosimilar to the primary epitope target, for example, in accordance with a BLOSUM62 scoring matrix.

Although performing a single binding reaction between a promiscuous affinity reagent and a complex protein sample, such as a human proteome sample, may yield ambiguous results regarding the identity of the different proteins to which it binds, the ambiguity can be resolved when the results are evaluated in a decoding method set forth herein. A plurality of binding outcomes obtained from measuring binding of a plurality of affinity reagents with one or more extant proteins can be input into a decoding method of the present disclosure to identify the most likely identity of that protein among a set of candidate proteins. The plurality of binding outcomes can be input into a decoding method along with information characterizing or identifying a plurality of candidate proteins (e.g. amino acid sequences of candidate proteins), and a binding model. The probability of each affinity reagent binding to every possible candidate protein can be evaluated using the binding model and the decoding method can output the identity of individual extant proteins. For example, the decoding algorithm can output the most likely identity for an individual extant protein as the candidate protein that is most compatible with the observed binding outcomes for the extant protein according to the binding model.

The present disclosure provides a decoding method, for example, in the form of a decoding algorithm, that can be used to evaluate the results of a binding reaction. The results can be used to identify, quantify or otherwise characterize macromolecules as exemplified herein for proteins. In some configurations, distinct and reproducible binding profiles may be observed for some or even a substantial majority of proteins that are to be identified in a sample. However, in many cases one or more binding events produces inconclusive or even aberrant results and this, in turn, can yield ambiguous binding profiles. For example, observation of binding outcomes at single-molecule resolution can be particularly prone to ambiguities due to stochasticity in the behavior of single molecules when observed individually. The present disclosure provides decoding methods that provide accurate protein identification despite ambiguities and imperfections that can arise in single-molecule formats or other contexts.

In some configurations, methods for identifying or characterizing one or more extant proteins utilize a decoding method that analyzes an empirical binding profile acquired for a plurality of binding reactions carried out between each extant protein and a plurality of affinity reagents, and then the empirical binding profile is evaluated with respect to the binding behavior of the affinity reagents to a plurality of candidate proteins. The decoding algorithm can output the identity of the extant protein as the candidate protein that has binding characteristics most compatible with the empirical binding profile. This compatibility can be determined based on a binding model that represents the affinity of each of the candidate proteins for each of the affinity reagents that were used to produce the empirical binding profile. A strong candidate protein can be identified as one for which the modeled binding outcomes are more consistent with the results of applying the binding model to the empirical binding profile as compared to the modeled binding outcomes for other candidate proteins evaluated. The decoding method can be configured to evaluate positive binding outcomes and, in some cases, negative binding outcomes. A weak candidate protein can be identified based on having many instances where positive binding outcomes and/or negative binding outcomes are inconsistent with the empirical binding profile being evaluated by a binding model. The strongest candidate protein can be deemed the most likely identity for the extant protein and confidence in this identification can be computed as a relative measure of the compatibility of the most likely protein compared to all the other candidate proteins.

A computer processor can be configured to execute a decoding method that outputs identities for one or more extant proteins based on various inputs. A particularly useful input is empirical binding data for binding of an extant protein to a plurality of different affinity reagents. The binding data can be in the form of an empirical binding profile that includes a plurality of empirically observed binding outcomes. An empirical binding profile can include positive binding outcomes or negative binding outcomes. The same can be true for a candidate outcome profile. In some configurations a binding profile will include both positive binding outcomes and negative binding outcomes. For example, decoding can be carried out in an ‘uncensored’ configuration, wherein both positive and negative binding outcomes are considered. Alternatively, decoding can be carried out in a ‘censored’ configuration, wherein a subset of binding outcomes or a particular type of binding outcome is not considered. For example, a censored configuration can consider positive binding outcomes and omit negative binding outcomes. A censored approach can be useful, for example, in situations where there is an expectation that particular binding measurements or binding outcomes are prone to an unacceptable or undesirable level of errors or artifacts.

An empirical binding profile can be input to a decoding method set forth herein. For example, the empirical binding profile can be input to a computer processor that performs the decoding method. A series of empirical binding outcomes that constitute an empirical binding profile can be acquired from detection of binding reactions such as those set forth herein or known in the art. Alternatively, a binding profile can be obtained from a simulation and used similarly to an empirical binding profile. Each empirical binding outcome in a binding profile can result from one binding reaction among a plurality of binding reactions carried out between a protein and a plurality of affinity reagents.

Another useful input to a decoding method is information for a plurality of candidate proteins. For example, a database of candidate protein information can be input to a computer processor that performs the decoding method. A plurality of candidate proteins may include at least 10, 25, 50, 75, 100, 500, 1×10³, 1×10⁴, 1×10⁶, 1×10⁸ or more different candidate proteins. In some cases, a complete proteome or substantial fraction thereof can be included. For example, a database can include at least 10%, 25%, 50%, 75%, 90%, 95%, 99% or more of the proteins known, or suspected, to be present in a proteome set forth herein or known in the art. In some embodiments, primary structures (i.e. amino acid sequences), secondary structures, tertiary structures, quaternary structures, names, or other information pertaining to the candidate proteins can be stored in a database. Particularly useful information that can be stored in a database includes, for example, binding characteristics for binding of one or more affinity reagents to a protein. However, such information need not be included and can instead be provided by a binding model. For example, the information can include a probability for each of a plurality of affinity reagents binding to each of a plurality of candidate proteins. A database can include a probability or likelihood that a candidate protein would generate a positive binding outcome. A database can further include a probability or likelihood that a candidate protein would generate a negative binding outcome. Any of a variety of databases can be used, such as those set forth in Egertson et al., BioRxiv (2021), DOI: 10.1101/2021.10.11.463967, US Pat. App. Pub. No. 2023/0114905 A1 or U.S. Pat. App. Ser. No. 63/254,420, each of which is incorporated herein by reference.

A binding model can be input to a decoding method set forth herein. For example, the binding model can be input to a computer processor that performs the decoding method. Optionally, a binding model can include a function for determining probability of a specific binding event occurring between a protein epitope and each of a plurality of affinity reagents. Epitopes evaluated by the model can have any of a variety of characteristics of interest. For example, the epitopes can have a defined length (e.g. the epitope length being less than or equal to 2, 3, 4, 5 or 6 amino acids in a protein primary sequence) or chemical composition (e.g. sequence of amino acids in a protein primary sequence). In some cases, the chemical composition can be relatively general with regard to chemical characteristics of amino acid side chains (or other moieties) such as charge, polarity, hydropathy, steric size, steric shape or the like. For example, the chemical composition of an epitope can be expressed in terms of biosimilarity to another epitope. In some cases, the probability of a specific binding event occurring between a protein epitope and an affinity reagent can depend on the presence or absence of a post-translational modification to the epitope. For example, an affinity reagent can have a higher probability of binding a post-translationally modified variant of a given protein epitope compared to its probability of binding a non-modified version of the given epitope. Alternatively, an affinity reagent can have a higher probability of binding a non-modified variant of a given protein epitope compared to its probability of binding a post-translationally modified version of the given epitope.

A decoding method set forth herein can include a function for calculating a probability of each affinity reagent binding to some or all possible candidate proteins among a plurality of candidate proteins in a given database. The function can consider positive binding outcomes. Optionally, the function can further consider negative binding outcomes, for example, when the function is used in an uncensored configuration. Optionally, binding probabilities can be configured as a matrix. As demonstrated in Egertson et al., BioRxiv (2021), DOI: 10.1101/2021.10.11.463967, US Pat. App. Pub. No. 2023/0114905 A1 or U.S. Pat. App. Ser. No. 63/254,420, positive binding outcomes can be included in an M×N binding probability matrix B.

A parameterized binding model can be used in a decoding method of the present disclosure. For example, an affinity reagent can be modeled by assigning a binding probability to each unique target epitope recognized by the affinity reagent. Optionally, a non-specific binding rate can be assigned to individual affinity reagents. The non-specific binding rate can, for example, represent probability of a given affinity reagent binding to any epitope in a protein non-specifically. The probability of an affinity reagent binding to a given candidate protein can be computed by first computing the probability of a specific binding event happening. The model can consider the count of each epitope in a given protein sequence. The binding model parameters can include a vector of probabilities of a given affinity reagent binding to each recognized epitope. Furthermore, the model can include a function for computing the probability of a non-specific protein binding event happening. Optionally, the model can take into account the length of each candidate protein sequence, the length of an epitope recognized by the affinity reagent or both. The probability of the affinity reagent binding to the protein and generating a detectable signal can be represented as the probability of one or more specific or non-specific binding events occurring. Exemplary binding models are provided in Egertson et al., BioRxiv (2021), DOI: 10.1101/2021.10.11.463967, US Pat. App. Pub. No. 2023/0114905 A1 or U.S. Pat. App. Ser. No. 63/254,420, each of which is incorporated herein by reference.

In some configurations of a system or method set forth herein, a non-specific binding rate can be provided as an input. The input can be in the form of one fixed non-specific binding rate for all affinity reagents, or a unique non-specific binding rate for each affinity reagent. Also, non-specific binding rate can be learned iteratively and/or adaptively in the same manner as other parameters in an affinity reagent binding model. The non-specific binding event can be binding of an affinity reagent to a substance other than a protein. The substance can be a solid support attached to an extant protein. For example, a non-specific binding event can occur at a region of an array where no protein of interest resides, such as a location at or near an address where a protein of interest resides. In some cases, a non-specific binding event can occur at an empty address, where a protein does not reside or at an interstitial region on the array that separates one address from another. Optionally, as exemplified in Egertson et al., BioRxiv (2021), DOI: 10.1101/2021.10.11.463967, US Pat. App. Pub. No. 2023/0114905 A1 or U.S. Pat. App. Ser. No. 63/254,420, the input can be a surface non-specific binding rate describing the probability of a surface non-specific binding event happening in any given cycle in a series of binding reactions.

Execution of a decoding algorithm can include computing a probability matrix that includes the probabilities of a positive binding outcome for individual affinity reagents binding to each candidate protein used in a binding reaction. Optionally, the method can further include computing a probability matrix that includes the probabilities of a negative binding outcome for individual affinity reagents binding to each candidate protein used in a binding reaction. For example, adjusted non-binding probabilities can be computed as set forth in Egertson et al., BioRxiv (2021), DOI: 10.1101/2021.10.11.463967, US Pat. App. Pub. No. 2023/0114905 A1 or U.S. Pat. App. Ser. No. 63/254,420, each of which is incorporated herein by reference. In an alternative configuration of systems and methods set forth herein, the probabilities of a negative binding outcome can be calculated by subtracting the probabilities of a positive binding outcome from 1, the probabilities being represented by a value between 0 and 1. Positive and negative binding outcomes can be equally weighted. Alternatively, positive binding outcomes can be weighted more heavily relative to negative binding outcomes. In other cases, negative binding outcomes can be weighted more heavily relative to positive binding outcomes. The latter weighting can be particularly desirable to account for the numerous difficult-to-predict mechanisms by which an affinity reagent may bind to proteins non-specifically.

Decoding can be carried out by computing a vector of likelihoods for a plurality of candidate proteins. The protein of highest likelihood can be selected. For example, the selected candidate protein can be the one having the most probabilities for binding the affinity reagents that are consistent with most of the binding outcomes obtained for a given extant protein. In another example, a candidate protein can be selected by multiplying the probabilities of the observed binding outcomes. Optionally, if there is a tie for top protein, one of the top proteins can be selected randomly or by another desired criteria. The probability of an identification being correct can be based on the likelihood of the top protein being correct divided by the sum of the likelihood of all other candidate proteins being correct. The protein identity can be output from the decoding system or method. Optionally, the probability of an identification being correct can be output. The probability can be calculated as the quotient of dividing the likelihood of a selected candidate protein by the sum of the likelihoods determined for all the other candidate proteins that were evaluated by the decoding algorithm.

Exemplary algorithms, and methods for characterizing proteins that can be used in combination with a method or system set forth herein include, for example, those set forth in US Pat App. Pub. No 2020/0286584 A1, US Pat. App. Pub. No. 2023/0114905 A1, U.S. Pat. App. Ser. No. 63/254,420 or Egertson et al., BioRxiv (2021), DOI: 10.1101/2021.10.11.463967, each of which is incorporated herein by reference.

A decoding method can output information pertaining to the identity for one or more extant proteins. The information output for a given protein can be in the form of a determined identity for the protein or in the form of a probability or likelihood for one or more identity of the protein. For example, the most likely identity for an extant protein, the likelihood or probability of the extant protein having a particular identity, or both can be output by a decoding method. A decoding method can output a non-digital or non-binary score for the identity of a given extant protein or for the likelihood of the extant protein having a particular identity. For example, probability or likelihood scores can be output in the form of an analog value between 0 and 1, or percent value between 0% and 100%. In some configurations, a digital or binary score that indicates one of two discrete states can be output to indicate the identity of a protein or at least a subset of proteins (e.g. a family of proteins sharing a common structural motif) to which the protein belongs.

Many macromolecule detection methods, such as enzyme linked immunosorbent assay (ELISA), can be configured to achieve high-confidence characterization of one or more macromolecules (e.g. one or more proteins) in a sample by performing binding reactions that exploit high specificity binding of antibodies, aptamers or other binding agents to the macromolecule(s) and detecting the binding event while ignoring all other analytes in the sample. ELISA is generally carried out at low plex scale (e.g. from one to a hundred different proteins detected in parallel or in succession) but can be used at higher plexity. ELISA methods can be carried out by detecting immobilized binding agents and/or macromolecules in multiwell plates, on arrays, or on particles in microfluidic devices. Exemplary plate-based methods include, for example, the MULTI-ARRAY technology commercialized by MesoScale Diagnostics (Rockville, Maryland) or Simple Plex technology commercialized by Protein Simple (San Jose, CA). Exemplary, array-based methods include, but are not limited to those utilizing Simoa ° Planar Array Technology or Simoa ° Bead Technology, commercialized by Quanterix (Billerica, MA). Further exemplary array-based methods are set forth in U.S. Pat. Nos. 9,678,068; 9,395,359; 8,415,171; 8,236,574; or 8,222,047, each of which is incorporated herein by reference. Exemplary microfluidic detection methods include those commercialized by Luminex (Austin, Texas) under the trade name xMAP® technology or used on platforms identified as MAGPIX®, LUMINEX® 100/200 or FEXMAP 3D®.

Other assays and reactions that can be used to detect proteins or other macromolecules, for example at low plex scale, include procedures that employ SOMAmer reagents and SOMAscan assays commercialized by Soma Logic (Boulder, CO). In one configuration, a sample is contacted with aptamers that are capable of binding proteins with specificity for the amino acid sequence of the proteins. The resulting aptamer-protein complexes can be separated from other sample components, for example, by attaching the complexes to beads (or other solid support) that are removed from other sample components. The aptamers can then be isolated and, because the aptamers are nucleic acids, the aptamers can be detected using any of a variety of methods known in the art for detecting nucleic acids, including for example, hybridization to nucleic acid arrays, PCR-based detection, or nucleic acid sequencing. The methods can be used to detect macromolecules other than proteins. Exemplary methods and compositions are set forth in U.S. Pat. Nos. 7,855,054; 7,964,356; 8,404,830; 8,945,830; 8,975,026; 8,975,388; 9,163,056; 9,938,314; 9,404,919; 9,926,566; 10,221,421; 10,239,908; 10,316,321 10,221,207 or 10,392,621, each of which is incorporated herein by reference.

In some assays and reactions, a polymeric macromolecule can be cyclically modified and the modified products from individual cycles can be detected. For example, a protein can be sequenced by a cyclical reaction in which each cycle includes steps of detecting the protein and removing one or more terminal amino acids from the protein. Optionally, one or more of the steps can include adding a label to the polymer, for example, at the amino terminal amino acid or at the carboxy terminal amino acid of a protein. In particular configurations, a method of detecting a protein can include steps of (i) exposing a terminal amino acid on the protein; (ii) detecting a change in signal from the protein; and (iii) identifying the type of amino acid that was removed based on the change detected in step (ii). The terminal amino acid can be exposed, for example, by removal of one or more amino acids from the amino terminus or carboxyl terminus of the protein. Steps (i) through (iii) can be repeated to produce a series of signal changes that is indicative of the sequence for the protein. Similar methods can be applied to other macromolecules by stepwise removal of monomers from an end of the polymer and detection of the modified polymer.

In a first configuration of a cyclical protein detection method, one or more types of amino acids in the protein can be attached to a label that uniquely identifies the type of amino acid. In this configuration, the change in signal that identifies the amino acid can be loss of signal from the respective label. For example, lysines can be attached to a distinguishable label such that loss of the label indicates removal of a lysine. Alternatively or additionally, other amino acid types can be attached to other labels that are mutually distinguishable from lysine and from each other. For example, lysines can be attached to a first label and cysteines can be attached to a second label, the first and second labels being distinguishable from each other. Exemplary compositions and techniques that can be used to remove amino acids from a protein and detect signal changes are those set forth in Swaminathan et al., Nature Biotech. 36:1076-1082 (2018); or U.S. Pat. Nos. 9,625,469 or 10,545,153, each of which is incorporated herein by reference. Methods and apparatus under development by Erisyon, Inc. (Austin, TX) may also be useful for detecting proteins.

In some configurations of a cyclical protein detection method, a terminal amino acid of a protein can be recognized by an affinity agent that is specific for the terminal amino acid or specific for a label moiety that is present on the terminal amino acid. The affinity agent can be detected on the array, for example, due to a label on the affinity agent. Optionally, the label is a nucleic acid barcode sequence that is added to a primer nucleic acid upon formation of a complex. For example, a barcode can be added to the primer via ligation of an oligonucleotide having the barcode sequence or polymerase extension directed by a template that encodes the barcode sequence. The formation of the complex and identity of the terminal amino acid can be determined by decoding the barcode sequence. Multiple cycles can produce a series of barcodes that can be detected, for example, using a nucleic acid sequencing technique. Exemplary affinity agents and detection methods are set forth in US Pat. App. Pub. No. 2019/0145982 A1; 2020/0348308 A1; or 2020/0348307 A1, each of which is incorporated herein by reference. Methods and apparatus under development by Encodia, Inc. (San Diego, CA) may also be useful for detecting proteins.

Cyclical removal of terminal amino acids from a protein can be carried out using an Edman-type sequencing reaction in which a phenyl isothiocyanate reacts with a N-terminal amino group under mildly alkaline conditions (e.g. about pH 8) to form a cyclical phenylthiocarbamoyl Edman complex derivative. The phenyl isothiocyanate may be substituted or unsubstituted with one or more functional groups, linker groups, or linker groups containing functional groups. An Edman-type sequencing reaction can include variations to reagents and conditions that yield a detectable removal of amino acids from a protein terminus, thereby facilitating determination of the amino acid sequence for a protein or portion thereof.

Edman-type processes can be carried out in a multiplex format to detect, characterize or identify a plurality of proteins. A method of detecting a protein can include steps of (i) exposing a terminal amino acid on a protein at an address of an array; (ii) binding an affinity agent to the terminal amino acid, wherein the affinity agent includes a nucleic acid tag, and wherein a primer nucleic acid is present at the address; (iii) extending the primer nucleic acid, thereby producing an extended primer having a copy of the tag; and (iv) detecting the tag of the extended primer. The terminal amino acid can be exposed, for example, by removal of one or more amino acids from the amino terminus or carboxyl terminus of the protein. Steps (i) through (iv) can be repeated to produce a series of tags that is indicative of the sequence for the protein. The method can be applied to a plurality of proteins on the array and in parallel. Whatever the plexity, the extending of the primer can be carried out, for example, by polymerase-based extension of the primer, using the nucleic acid tag as a template. Alternatively, the extending of the primer can be carried out, for example, by ligase- or chemical-based ligation of the primer to a nucleic acid that is hybridized to the nucleic acid tag. The nucleic acid tag can be detected via hybridization to nucleic acid probes (e.g. in an array), amplification-based detections (e.g. PCR-based detection, or rolling circle amplification-based detection) or nuclei acid sequencing (e.g. cyclical reversible terminator methods, nanopore methods, or single molecule, real time detection methods). Exemplary methods that can be used for detecting proteins using nucleic acid tags are set forth in US Pat. App. Pub. No. 2019/0145982 A1; 2020/0348308 A1; or 2020/0348307 A1, each of which is incorporated herein by reference.

A macromolecule can optionally be detected based on a reaction that measures function or activity. For example, a protein can optionally be detected based on its enzymatic activity or other measurable biological activity. In some configurations of the present methods, a protein can be contacted with a reactant that is converted to a detectable product by an enzymatic activity of the protein. In other assay formats, a first protein having a known enzymatic function can be contacted with a second protein to determine if the second protein changes the enzymatic function of the first protein. As such, the first protein serves as a reporter system for detection of the second protein. Exemplary changes that can be observed using a functional assay or reaction include, but are not limited to, activation of the enzymatic function, inhibition of the enzymatic function, attenuation of the enzymatic function, degradation of the first protein or competition for a reactant or cofactor used by the first protein. Proteins can also be assayed using a reaction that detects their binding interactions with other molecules such as proteins, nucleic acids, nucleotides, metabolites, hormones, vitamins, small molecules that participate in biological signal transduction pathways, biological receptors or the like. For example, a protein that participates in a signal transduction pathway can be identified as a particular candidate protein by detecting binding to a second protein that is known to be a binding partner for the candidate protein in the pathway. Typically, proteins will be in a native conformation when subjected to a functional or activity assay. However, denatured proteins can be subjected to a functional or activity assay, for example, by way of comparison to an assay performed on the proteins in a native conformation.

A protein or other macromolecule can be detected based on proximity of two or more bound affinity reagents. For example, the two affinity reagents can include two components each: a receptor component and a nucleic acid component. When the affinity reagents bind in proximity to each other in a binding assay or reaction, for example, due to epitopes for the respective affinity reagents being on a single protein, or due to the epitopes being present on two proteins that associate with each other, the nucleic acids can interact to cause a modification that is indicative of the two epitopes being in proximity. Optionally, the modification can be polymerase-catalyzed extension of one of the nucleic acids using the other nucleic acid as a template. As another option, one of the nucleic acids can form a template that acts as splint to position other nucleic acids for ligation to an oligonucleotide. Exemplary methods are commercialized by Olink Proteomics AB (Uppsala Sweden) or set forth in U.S. Pat. Nos. 7,306,904; 7,351,528; 8,013,134; 8,268,554 or 9,777,315, each of which is incorporated herein by reference.

A reaction can be used to modify an analyte such as a protein or other macromolecule. For example, a polymeric macromolecule can be selectively modified at one or more type of monomer. The modification can be detectable, for example, due to the introduction of a label moiety to the macromolecule. Taking proteins as an example, one or more type of amino acid can be modified, for example, to introduce a label moiety. Exemplary types of amino acids having side chains that can be labeled include, but are not limited to, serine, threonine, tyrosine, histidine, cysteine, lysine, aspartate, glutamate, asparagine or proline. Any of a variety of labels can be used including, for example, those set forth herein.

A method of the present disclosure can include a step of determining the extent of reaction between one or more macromolecules and an assay reagent. In particular configurations of the methods set forth herein, reaction extent can be determined as the quantity of macromolecules subjected to an assay reagent that yield a given measurement outcome relative to the quantity of the macromolecules that do not yield the given measurement outcome. For example, reaction extent can be determined as the quantity of macromolecules that yield a positive measurement outcome when subjected to an assay reagent relative to the quantity of the macromolecules that yield a negative measurement outcome. In some cases, reaction extent can be determined as the quantity of macromolecules that yield a positive measurement outcome when subjected to an assay reagent relative to both the quantity of the macromolecules that yield a negative measurement outcome and the quantity of the macromolecules that yield a null measurement outcome. The measurement outcome can be any of a variety of outcomes acquired from a macromolecule assay. For example, binding outcomes can be acquired from a binding assay set forth herein, such as an assay the measures binding of affinity reagents to proteins. As such, binding extent (also referred to as “binding rate” herein) can be determined as the quantity of proteins in a binding assay that bind to a particular affinity reagent relative to the quantity of proteins in the binding assay that do not bind to the affinity reagents.

In multiplex formats, such as those configured to detect a plurality of different macromolecules, reaction extent can be determined for a particular type of macromolecule. Taking as an example a multiplex protein binding assay, protein molecules that are identified as having the same primary structure (i.e. amino acid sequence) can be evaluated with respect to whether or not they bind to a particular affinity reagent. In this example, binding extent can be determined as the quantity of the proteins that are considered to be of the designated type and that bind to a given affinity reagent in a binding assay relative to the amount of proteins of the designated type that do not bind to the given affinity reagent in the assay. Alternatively, binding extent can be determined as the quantity of the proteins that are considered to be of the designated type and that bind to a given affinity reagent in a binding assay relative to the total amount of proteins of the designated type (i.e. including those that do bind to the given affinity reagent and those that do not bind to the given affinity reagent). The type of macromolecule present in an assay, for example, the type present at each of a plurality of addresses in an array, can be identified using a method set forth herein. For example, proteins can be identified at addresses of an array, and proteins identified as having the same amino acid sequence can be evaluated for reaction extent with one or more assay reagents.

In an exemplary configuration, a method of the present disclosure can include steps of (a) contacting an array of macromolecules with a plurality of different assay reagents, wherein individual addresses of the array are each attached to a single macromolecule, the macromolecule having a plurality of different reactive sites, and wherein the different assay reagents are different with respect to specificity for the reactive sites; (b) detecting reaction of the array of macromolecules with the different assay reagents, whereby the individual addresses of the array are resolved, and whereby the different assay reagents are resolved; and (c) determining a first observed reaction extent comprising the fraction of the individual addresses having a first reactive site that is observed to react with a first assay reagent of the plurality of different assay reagents in step (b). Optionally, the observed reaction extent includes the fraction of the individual addresses observed to have a positive measurement outcome with the assay reagent in step (b) relative to the fraction of the individual addresses observed to have a negative measurement outcome with the assay reagent in step (b). Alternatively, the observed reaction extent includes the fraction of the individual addresses observed to have a particular macromolecule type and a positive measurement outcome with the assay reagent in step (b) relative to the number of the individual addresses observed to have the particular macromolecule type.

In a second exemplary configuration, a method of the present disclosure can include steps of (a) contacting an array of macromolecules with a plurality of different assay reagents, wherein individual addresses of the array are each attached to a single macromolecule; (b) detecting reaction of the array of macromolecules with the different assay reagents; (c) determining a first observed reaction extent including the fraction of the individual addresses observed to react with a first assay reagent in step (b); and (d) determining a second observed reaction extent including the fraction of the individual addresses observed to react with a second assay reagent in step (b). Optionally, the single reaction extent observed for the individual address reacting with an assay reagent includes the fraction of the individual addresses observed to have a positive measurement outcome with the first assay reagent in step (b) relative to the fraction of the individual addresses observed to have a negative measurement outcome with both the first assay reagent and the second assay reagent in step (b). Optionally, the single reaction extent includes the fraction of the individual addresses having a particular macromolecule type and a positive measurement outcome with the assay reagent in step (b) relative to the number of the individual addresses observed to have the particular macromolecule type.

When determining reaction extents, macromolecules can be categorized into types according to any of a variety of desired characteristics and the number of macromolecules of one or more types can be quantified using methods, compositions or systems set forth herein. For example, proteins can be categorized as being the same type due to (1) having the same full length amino acid sequence, (2) having the same full length amino acid sequence whether or not the proteins differ with respect to one or more post-translational modification(s), (3) having the same full length amino acid sequence and the same type(s) of post-translational modification(s), (4) having the same full length amino acid sequence and having post-translational modification(s) at the same amino acid position(s), (5) having the same full length amino acid sequence and having post-translational modification(s) of the same type and at the same amino acid position(s), (6) having a domain with the same amino acid sequence, (7) having a domain with the same amino acid sequence whether or not one or more other domains of the full length sequences differ, (8) having a domain with the same amino acid sequence whether or not the domains differ with respect to post-translational modification(s), (9) having a domain with the same amino acid sequence and the same type(s) of post-translational modification(s), (10) having a domain with the same amino acid sequence and post-translational modification(s) at the same amino acid position(s) in the domain, or (11) having a domain with the same amino acid sequence and post-translational modification(s) of the same type and at the same amino acid position(s) in the domain. Other useful characteristics upon which proteins can be designated as being the same type include, but are not limited to, having the same number of amino acids in the full-length sequence; having the same molecular weight or mass; having an epitope for a given affinity reagent or group of affinity reagents; having the same solubility in aqueous solutions, such as a solution that mimics a biological fluid, cell cytosol or other milieu; having the same or different solubility in non-aqueous solutions, such as a lipid, oil or solution that mimics a cell membrane; or a combination of the foregoing.

Single molecule assays can be particularly useful for quantifying proteins or other macromolecules. Taking as an example an assay performed on a single-molecule array having only one protein per unique identifier, counting of the unique identifiers to which a particular type of protein is attached, can be used to quantify proteins of that type in the sample. In some configurations, a first subset of unique identifiers in an array is attached to one protein type and only one protein type. In this configuration, a second subset of unique identifiers may lack any protein, and/or a third subset of unique identifiers may be attached to more than one protein type. Methods set forth herein, or particular steps of the methods, can be configured to include one or more of the first, second or third subset of unique identifiers. Alternatively, methods set forth herein, or particular steps of the methods, can be configured to exclude one or more of the first, second or third subset of unique identifiers.

Macromolecules can be quantified using assays that detect ensembles such as array-based assays, in which individual addresses of the array each contain an ensemble of macromolecules of the same type. The macromolecules can be quantified based on the count of the ensembles or based on a count of macromolecules within one or more ensemble. Depending upon the assay format used, macromolecules of a given type can be quantified based on bulk-level or ensemble-level measures such as concentration of the macromolecules, total mass of the macromolecules, activity of the macromolecules, total number of epitopes or other reactive sites of a given type, or other measures known in the art.

As exemplified above, a reaction extent can be determined for reaction of a single type of assay reagent with a given type of macromolecule. Alternatively or additionally, a method set forth herein can include a step of determining the extent to which two types of assay reagent react with a given type of macromolecule. For example, a double reaction extent can be determined as the fraction of macromolecules (e.g. proteins) of a given type that react with a two different types of assay reagent. More than two different assay reagents can be used to determine a multiple reaction extent. The plurality of different types of assay reagent can include, for example, at least 2, 3, 4, 5, 6, 7, 8, 9 10 or more different types of assay reagent. Accordingly, a method set forth herein can include a step of determining an n-tuple reaction extent that is at least a double (n=2) reaction extent, triple (n=3) reaction extent, quadruple (n=4) reaction extent, quintuple (n=5) reaction extent, sextuple (n=6) reaction extent, septuple (n=7) reaction extent, octuple (n=8) reaction extent, nonuple (n=9) reaction extent, decuple (n=10) reaction extent, or higher n-tuple reaction extent.

Multiple reactions performed in a method set forth herein need not be simultaneous and can instead occur sequentially. Products of the multiple reactions can be detected simultaneously whether or not the reactions occur simultaneously. If desired, products of the multiple reactions can be detected serially. In the case of a binding assay, a multiple binding extent can be determined, for example, as the fraction of macromolecules (e.g. proteins) of a given type that bind to multiple different types of affinity reagent. The binding events can occur simultaneously or sequentially, and binding outcomes can be detected simultaneously or sequentially. A particularly useful configuration of the methods set forth herein, can utilize two different types of assay reagents to determine a double reaction extent. For example, in the case of a binding assay two different types of affinity reagents can be used to determine a double binding extent. A double reaction extent, such as a double binding extent, can provide for convenient characterization of macromolecule structures using pairwise comparison of assay results.

In an exemplary configuration, a method of the present disclosure can include steps of (a) contacting an array of macromolecules with a plurality of different assay reagents, wherein individual addresses of the array are each attached to a single macromolecule; and (b) detecting reaction of the array of macromolecules with the different assay reagents, (c) determining a first observed reaction extent including the fraction of the individual addresses observed to react with a first assay reagent in step (b); (d) determining a second observed reaction extent including the fraction of the individual addresses observed to react with a second assay reagent in step (b), and (e) determining an observed double reaction extent including the fraction of the individual addresses observed to react with both the first assay reagent and the second assay reagent in step (b). Optionally, the reaction extent observed for the individual address reacting with an assay reagent can include the fraction of the individual addresses observed to have a positive measurement outcome with the first assay reagent in step (b) relative to the fraction of the individual addresses observed to have a negative measurement outcome with both the first assay reagent and the second assay reagent in step (b). Optionally, the reaction extent observed for the individual address reacting with an assay reagent can include the fraction of the individual addresses having a particular macromolecule type and a positive measurement outcome with the first assay reagent in step (b) relative to the number of the individual addresses observed to have the particular macromolecule type. As a further option the observed double reaction extent can include the fraction of the individual addresses observed to have positive measurement outcomes with both the first assay reagent and the second assay reagent relative to the fraction of the individual addresses observed to have a negative measurement outcome with both the first assay reagent and the second assay reagent. Optionally, the observed double reaction extent can include the fraction of the individual addresses having a particular macromolecule type and a positive measurement outcome with both the first and second assay reagent relative to the number of the individual addresses observed to have the particular macromolecule type.

A reaction extent can be determined from an empirical observation such as a measurement outcome from an assay or reaction set forth herein. In some cases, an ‘expected’ reaction extent can be determined, for example, from a theoretical model or statistical model. Similarly, an observed multiple reaction extent can be determined from the fraction of macromolecules of a given type in a reaction that is observed to react with multiple different types of assay reagent. For example, an observed double reaction extent can be determined from the fraction of macromolecules of a given type that is observed to react with a first assay reagent and a second assay reagent, wherein the first assay reagent is a different type from the second assay reagent. The assay reagent types can differ with respect to their specificity for reactive sites in a given macromolecule. For example, the different assay reagent types can be antibodies (or other affinity reagents) that bind to different epitopes.

In an exemplary configuration, a method of the present disclosure can include steps of (a) contacting an array of macromolecules with a plurality of different assay reagents, wherein individual addresses of the array are each attached to a single macromolecule; and (b) detecting reaction of the array of macromolecules with the different assay reagents, and (c) determining an observed double reaction extent including the fraction of the individual addresses observed to react with both the first assay reagent and the second assay reagent. Optionally, the observed double reaction extent can be determined as the fraction of the individual addresses observed to have positive measurement outcomes with both the first assay reagent and the second assay reagent relative to the fraction of the individual addresses observed to have a negative measurement outcome with both the first assay reagent and the second assay reagent. Optionally, the observed double reaction extent includes the fraction of the individual addresses observed to have a particular macromolecule type and positive measurement outcomes with the first and second assay reagent in step (b) relative to the number of the individual addresses observed to have the particular macromolecule type.

A method of the present disclosure can include a step of determining accessibility of a reactive site of a macromolecule based on a comparison of an observed reaction extent and an expected reaction extent. For example, a method set forth herein can include steps of (a) contacting an array of macromolecules with a plurality of different assay reagents, wherein individual addresses of the array are each attached to a single macromolecule, the macromolecule having a plurality of different reactive sites, and wherein the different assay reagents are different with respect to specificity for the reactive sites; (b) detecting reaction of the array of macromolecules with the different assay reagents, whereby the individual addresses of the array are resolved, and whereby the different assay reagents are resolved; (c) determining a first observed reaction extent comprising the fraction of the individual addresses having a first reactive site that is observed to react with a first assay reagent of the plurality of different assay reagents in step (b); (d) providing an expected reaction extent including the extent to which the assay reagent reacts with a candidate macromolecule having the same molecular structure as the macromolecules at the fraction of the individual addresses observed to react with the first assay reagent in step (b); and (e) determining accessibility of the first reactive site of the macromolecules based on a comparison of the observed reaction extent and the expected reaction extent. Optionally, the expected reaction extent can be obtained from a database. As a further option, the expected reaction extent can be determined from an assay performed for the macromolecules. For example, the expected reaction extent can be obtained by (i) contacting a second array of macromolecules with the plurality of different assay reagents, wherein individual addresses of the second array are each attached to a candidate macromolecule having the same molecular structure as the macromolecules at the fraction of the individual addresses observed to react with the first assay reagent in step (b); (ii) detecting reaction of the second array of macromolecules with the different assay reagents, whereby the individual addresses of the array are resolved, and whereby the different assay reagents are resolved; (iii) determining the expected reaction extent including the fraction of the individual addresses of the second array having the first reactive site.

A second array that is used in a method set forth herein, such as a method configured as exemplified above, can include macromolecules that are obtained from the same source as the macromolecules present on one or more other arrays used in the method. For example, a biological sample can be divided into aliquots and macromolecules from individual aliquots can be attached to respective arrays. The aliquots can be treated similarly to provide replicate arrays. Alternatively, the aliquots can be subjected to different conditions or treatments. Optionally, aliquots can differ with regard to the kind of condition or treatment to which they are subjected. For example, aliquots can differ with regard to whether or not they are contacted with a therapeutic agent, drug candidate, metabolite, signaling molecule, vaccine, salt, organic solvent, carcinogen, pathogen, toxin or other biologically active substance; or whether they are exposed to a particular temperature, pH, redox state, osmolarity, radiation level, radiation wavelength, pressure, humidity or other physical condition. Alternatively, the aliquots can be subjected to the same condition or treatment, albeit to different degrees. For example, aliquots can be treated with differing amounts of a therapeutic agent, drug candidate, metabolite, signaling molecule, vaccine, salt, organic solvent, carcinogen, pathogen, toxin or other biologically active substance; or differing temperature, pH, redox state, osmolarity, radiation level, radiation wavelength, pressure, humidity or other physical condition.

A second array that is used in a method set forth herein, such as a method configured as exemplified above, can include macromolecules that are obtained from a different source compared to the source for the macromolecules present on one or more other arrays used in the method. For example, the macromolecules on the respective arrays can come from different cell types, different tissue types, different biological fluids, different cell cultures, different organs or the like. Alternatively or additionally, the macromolecules on the respective arrays can come from the same individual (i.e. having the same genome) or different individual (i.e. having different genomes). In some cases, the macromolecules on the respective arrays can come from samples that differ with respect to disease state. For example, a first sample can be known or suspected to be in a disease state and one or more other samples can be known or suspected to not be in the disease state.

A method set forth herein can be configured to determine observed reaction extents for a plurality of assay reagents toward a plurality of reactive sites. For example, a method can be configured to determine a first observed reaction extent for a first assay reagent and a macromolecule and a second observed reaction extent for a second assay reagent and the macromolecule. In this configuration, the method can include a step of obtaining a first expected reaction extent and a second expected reaction extent. In turn, accessibility can be determined for the reactive sites in the macromolecule to which the first and second assay reagents reacted, respectively. For example, accessibility of the first reactive site of the macromolecules can be determined based on a comparison of the first observed reaction extent and the first expected reaction extent; and accessibility of the second reactive site of the macromolecules can be determined based on a comparison of the second observed reaction extent and the second expected reaction extent. When the macromolecule is a polymer, the accessibility of the different reactive sites can be evaluated with respect to the locations of the reactive sites in the polymer. Looking to the example of proteins that are assayed using affinity reagents that recognize short peptide epitopes (e.g. epitopes composed of amino acid trimers), the position of the epitopes along the protein sequence can be determined. As such, the accessibility of those sequences, and perhaps the surrounding region of the protein sequence, can be determined.

Accessibility can be evaluated in relative terms. For example, accessibility of an observed macromolecule can be evaluated relative to accessibility of a candidate molecule having the same chemical structure as the observed macromolecule. For example, an observed protein can be evaluated with respect to a candidate protein having the same amino acid sequence as the observed protein. Moreover, expected reaction extent for one or more assay reagents reacting with particular reactive sites can be derived from the candidate protein. Potential outcomes for a relative accessibility determination include, for example, (1) a first reactive site in an observed macromolecule having lower than expected accessibility compared to the first reactive site in a candidate macromolecule, and a second reactive site in the observed macromolecule having lower than expected accessibility compared to the second reactive site in the candidate macromolecule; (2) a first reactive site in an observed macromolecule having lower than expected accessibility compared to the first reactive site in a candidate macromolecule, and a second reactive site in the observed macromolecule having accessibility that is substantially the same as the expected accessibility of the second reactive site in the candidate macromolecule; (3) a first reactive site in an observed macromolecule having higher than expected accessibility compared to the first reactive site in a candidate macromolecule, and a second reactive site in the observed macromolecule having higher than expected accessibility compared to the second reactive site in the candidate macromolecule; and (4) a first reactive site in an observed macromolecule having higher than expected accessibility compared to the first reactive site in a candidate macromolecule, and a second reactive site in the observed macromolecule having accessibility that is substantially the same as the expected accessibility of the second reactive site in the candidate macromolecule. Indeed, the above outcomes can be observed for more than two reactive sites in a macromolecule, thereby providing a characterization of accessibility for multiple regions of the macromolecule.

In some cases, a population of macromolecules may be observed to include two or more subpopulations, each subpopulation having different combinations of accessible and inaccessible reactive sites. The subpopulations can be identified, for example, using a cluster analysis or other technique for delineating subsets or groupings of datapoints in a larger dataset. The resulting grouping can be useful for determining the number of conformational states that the macromolecules achieve. Moreover, the locations of accessible and inaccessible reactive sites within the macromolecules in each cluster can provide insight regarding the three-dimensional structure of various regions in the chemical structure of the macromolecule. For example, the locations of accessible and inaccessible amino acids within the proteins of a given cluster can provide insight into the overall fold of the proteins in the cluster or the location of accessible and inaccessible domains in the proteins of a cluster. The methods set forth herein can be used to observe changes in the composition of clusters obtained for a given population of proteins, for example, due to different treatment of the proteins prior to being assayed, due to different assay conditions for the proteins, due to different sources for the proteins, or due to the use of different assay reagents for detecting the proteins.

A method of the present disclosure can include a step of determining accessibility of a reactive site of a macromolecule based on a comparison of an observed double reaction extent and an expected double reaction extent. As set forth herein, the observed double reaction extent can be configured as the fraction of macromolecules that are observed to react with both a first assay reagent and a second assay reagent in a reaction set forth herein. For example, the assay can include steps of (a) contacting an array of macromolecules with a plurality of different assay reagents, and (b) detecting reaction of the array of macromolecules with the different assay reagents, whereby the different assay reagents are resolved. The expected double reaction extent can be determined from a first observed reaction extent and a second observed reaction extent, wherein the first observed reaction extent is configured as the fraction of the individual addresses of the array that are observed to react with a first assay reagent in the assay, and wherein the second observed reaction extent is configured as the fraction of the individual addresses observed to react with a second assay reagent in the assay. Optionally, the macromolecule at each address of the array can have a plurality of different reactive sites, and the different assay reagents can be different with respect to specificity for the reactive sites. As a further option, individual addresses of the array are each attached to a single macromolecule, and the individual addresses of the array are individually resolved during the detecting step. Optionally, comparison of an observed double reaction extent and an expected double reaction extent can be configured as a ratio of the expected double reaction extent to the observed double reaction rate.

In some configurations of the methods set forth herein, a step of determining accessibility of a first reactive site of a macromolecule can be carried out by determining accessibility of the first reactive site relative to accessibility of a second reactive site of the macromolecule. Optionally, the method can further include determining accessibility of a first domain of the macromolecule relative to accessibility of a second domain of the macromolecule, wherein the first domain includes the first reactive site and the second domain includes the second reactive site.

A domain of a macromolecule can be identified in accordance with criteria known in the art. Taking proteins as an example, a domain can be delineated in terms of structure, function and/or evolution. A domain can be delineated in terms of a region of a polypeptide chain (a) that is self-stabilizing, (b) that folds independently from the rest of the polypeptide chain, and/or (c) that forms a compact folded three-dimensional structure. Optionally, a domain can be delineated in terms of a portion of a polypeptide chain that is expressed from a given exon, or a given subset of exons, in the gene that encodes the polypeptide chain. A domain can, in some cases, be delineated in terms of a region of a polypeptide that shares substantial amino acid sequence homology with a region of one or more other polypeptide. Two polypeptide sequence regions can be considered to have substantial homology if at least 30% of the amino acids are identical when the sequences are aligned, and the regions include at least 50 amino acids from each aligned sequence. It will be understood that more stringent criteria can be applied for aligned sequence regions if desired. For example, the length of each aligned region can be at least 75, 100, 150, 200, 250 or more amino acids. Moreover, the cutoff for substantial homology of two sequences in any of the aforementioned length ranges can be at least 40%, 50%, 60%, 70%, 80%, 90% or more of the aligned amino acids. A domain of a polypeptide can be delineated in terms of a region of the polypeptide chain that is capable of performing a particular biological function when isolated from the rest of the polypeptide chain. The biological function can be, for example, enzymatic function; binding specificity for a ligand, enzyme substrate, or protein; or susceptibility to being modified, for example, by a kinase or other regulatory enzyme. A domain of a polypeptide can be delineated in terms of a region of the polypeptide chain that has been more highly conserved through evolution compared to the rest of the polypeptide. Conservation can be determined according to homology, for example, as set forth above in the context of delineating structural domains.

In some configurations of the methods set forth herein, a step of determining accessibility of a first reactive site of a macromolecule can be carried out by determining relative accessibility for a first reactive site of the macromolecule compared to accessibility of two or more other reactive sites of the macromolecule. The use of multiple reactive sites can provide the advantage of increased accuracy or confidence for determining structural characteristics of protein domains. Accordingly, in a first option, the method can further include determining accessibility of a first domain of the macromolecule relative to accessibility of a second domain of the macromolecule, wherein the first domain includes the first reactive site and the second domain includes the two or more other reactive sites. As a second option, the method can further include determining accessibility of a first domain of the macromolecule relative to accessibility of a second domain of the macromolecule, wherein the first domain includes two or more reactive sites of the plurality of different reactive sites and the second domain includes two or more other reactive sites of the plurality of different reactive sites. For the second option, the accessibility of the first domain can be determined relative to the second domain by performing a plurality of relative accessibility determinations of nearest neighbor reactive sites in the primary molecular structure of the macromolecule.

Relative accessibility can be determined for a series of nearest neighbor reactive sites using a sliding window over the primary structure of a macromolecule. See, for example, FIG. 1. For proteins that are detected with affinity reagents, the sliding window can evaluate nearest neighbor epitopes of the affinity reagents to determine accessibility across the amino acid sequence of the protein. However, analysis of relative accessibility need not be limited to evaluating nearest neighbor reactive sites in the primary molecular structure of a macromolecule. For example, relative accessibility can be determined for all possible pairs of reactive sites in the primary molecular structure of the macromolecule. If desired, a first subset of reactive sites in a macromolecule can be subjected to a relative accessibility determination, such that another subset of reactive sites in the macromolecule is omitted from the determination. Hypotheses regarding the structure of the macromolecule, such as known or predicted domain structures in the macromolecule, can be used as a basis for choosing which pairs of reactive sites are subjected to the determination and which are omitted.

The present disclosure further provides a method of determining accessibility of macromolecule structures, including steps of (a) contacting an array of macromolecules with a plurality of different assay reagents, wherein individual addresses of the array are each attached to a single macromolecule, the macromolecule comprising a plurality of different reactive sites, and wherein the different assay reagents are different with respect to specificity for the reactive sites; (b) detecting reaction outcomes for individual addresses in the array with each of the different assay reagents, whereby the individual addresses of the array are resolved, whereby the different assay reagents are resolved, and wherein a collection of reaction outcomes for each of the addresses forms an empirical outcome profile; (c) comparing the empirical outcome profile for each of the addresses with a plurality of candidate outcome profiles, each of the candidate outcome profiles including a probability of a given candidate macromolecule reacting with the different assay reagents, thereby identifying a set of addresses having a similar candidate macromolecule; (d) identifying at least two subsets of the addresses having the similar candidate macromolecule, wherein the two subsets include different isoforms of the similar candidate macromolecule; and (e) evaluating reaction outcomes for the different isoforms to determine differential accessibility of a first reactive site in the different isoforms of the similar candidate macromolecule.

Empirical outcome profiles can be compared to candidate outcome profiles using a decoding method such as those set forth herein, or in US Pat. App. Pub. No. 2023/0114905 A1 or Egertson et al., BioRxiv (2021), DOI: 10.1101/2021.10.11.463967, each of which is incorporated herein by reference. The decoding method can identify macromolecules at addresses in an array. In many cases, an array will include instances of multiple addresses where similar macromolecules are present. For example, multiple addresses in a protein array can each be attached to a protein and the proteins at the addresses can have amino acid sequences that are identical to each other. Although decoding may identify the same macromolecule at a set of addresses, the addresses may have nonetheless produced empirical outcome profiles that differ from each other. In some formats of the methods set forth herein the observation of different empirical outcome profiles for the same protein may be expected or at least unsurprising. For example, assays that manipulate individual molecules or detect at single molecule resolution may produce stochastic results. As such, the empirical outcome profiles or decode output for a set of addresses, although pointing to the same candidate protein, may include random variations when compared to each other. However, evaluation of the empirical outcome profiles or decode output for the set of addresses may include sub-structures due to the addresses including two or more isoforms of a given macromolecule. For example, a set of addresses in a protein array may be decoded to indicate presence of the same amino acid sequence, but sub-structures in the empirical outcome profiles or decode output for the set of addresses may indicate that the set includes subsets of addresses attached to two or more proteoforms, respectively.

Subsets of addresses can be identified within a set of addresses by evaluating empirical outcome profiles or decode outputs using any of a variety of methods including, for example, clustering methods, such as k-means clustering hierarchical clustering or mixture models; generative statistical models, such as latent Dirichlet allocation or Bayesian networks; or signal separation such as principal component analysis, independent component analysis, non-negative matrix factorization or singular value decomposition. Unsupervised learning methods can be particularly useful for evaluating empirical outcome profiles or decode outputs to identify subsets of addresses within a set of addresses.

Once isoforms of a macromolecule have been identified, reaction outcomes for the different isoforms can be evaluated to determine differential accessibility of one or more reactive sites in the different isoforms. The evaluation can be carried out by: (i) representing each similar candidate macromolecule as a sequence of ordered reaction sites, (ii) determining probability that empirical reaction outcomes for the macromolecules correspond to individual reaction sites in the sequence of ordered reaction sites, and (iii) identifying a region of unlikely non-reaction of assay reagents in the sequence of ordered reaction sites. In some cases, the region of unlikely non-reaction includes a plurality of reaction sites in the sequence. Optionally a null distribution can be generated and compared to the identified region. As a further option a false discovery rate can be obtained. Further aspects of the method are set forth in Example I, herein.

In a particular configuration, a method of determining accessibility of macromolecule structures, can include steps of (a) contacting an array of proteins with a plurality of different affinity reagents, wherein individual addresses of the array are each attached to a single protein, the protein comprising a plurality of different epitopes, and wherein the different affinity reagents are different with respect to specificity for the epitopes; (b) detecting reaction outcomes for individual addresses in the array with each of the different affinity reagents, whereby the individual addresses of the array are resolved, whereby the different affinity reagents are resolved, and wherein a collection of reaction outcomes for each of the addresses forms an empirical outcome profile; (c) comparing the empirical outcome profile for each of the addresses with a plurality of candidate outcome profiles, each of the candidate outcome profiles including a probability of a given candidate protein reacting with the different affinity reagents, thereby identifying a set of addresses having a similar candidate protein; (d) identifying at least two subsets of the addresses having the similar candidate protein, wherein the two subsets include different proteoforms of the similar candidate protein; and (e) evaluating reaction outcomes for the different proteoforms to determine differential accessibility of a first epitope in the different isoforms of the similar candidate protein. Optionally, step (e) includes (i) representing the similar candidate protein as a sequence of ordered epitopes, (ii) determining probability that the empirical reaction outcomes correspond to individual epitopes in the sequence of ordered epitopes, and (iii) identifying a region of unlikely non-binding of affinity reagents in the sequence of ordered epitopes.

Methods set forth herein for determining accessibility of macromolecules can utilize samples having a variety of different macromolecules and multiple copies of each different macromolecule. For example, a plurality of proteins in a sample can include a variety of different primary structures (e.g. protein products expressed from a variety of different genes) and the plurality can further include multiple copies of each primary structure. The accessibility of a first set of copies (i.e. the set of copies for a first primary structure) in a sample can differ from the accessibility of a second set of copies (i.e. the set of copies for a second primary structure) in the sample. As such, heterogeneity in accessibility can be apparent for the proteins in the sample. Samples with a high degree of heterogeneity in accessibility are particularly well suited to the methods set forth herein.

Macromolecule accessibility can be determined to benefit any of a variety of applications. An exemplary application is to evaluate reagents or conditions used for assaying macromolecules. For example, a sample of macromolecules can be subjected to a first assay that employs a first condition or reagent, and the results can be evaluated to determine a level of accessibility of one or more macromolecules in the first assay. A second assay can be performed using the sample (or a replicate of the sample), a condition or reagent used in the second assay differing from the first assay, and the results can be evaluated to determine a level of accessibility of the one or more macromolecules in the second assay. A finding of low accessibility for a given macromolecule using a particular assay condition or reagent can indicate that another condition or reagent would be preferred as an alternative. The level of accessibility for a plurality of macromolecules in both assays can be compared to determine the degree of heterogeneity in accessibility for the two assays. In some situations, it may be desirable to reduce the degree of heterogeneity, for example, to favor confidence in identifying different macromolecules in a sample or quantifying the levels of different macromolecules in the sample.

A variety of conditions and reagents for detecting proteins can be tested and the results can be evaluated to determine differences in accessibility for reactive sites in one or more proteins in the tests. Results for a single protein type (e.g. one or more copies expressed from a given gene) can be evaluated to identify an assay reagent that reacts with a more accessible reactive site of the protein. The assay can be modified to use the identified assay reagent as an alternative or addition to an assay reagent that targeted a reactive site having relatively low accessibility. Another response may be to deploy altered assay conditions that improve accessibility of the reactive site. For example, the protein can be exposed to denaturing conditions that differ from the conditions used in the original assay. Yet another response may be to re-evaluate assay results. For example, parameters or variables used in a decode method can be adjusted based on the observed accessibility of one or more reactive sites. Exemplary parameters or variables that can be adjusted include, but are not limited to, binding probability of a particular affinity reagent for one or more candidate proteins, threshold for a false discovery rate, severity or nature of random noise assumed in a binding model, severity or nature of experimental confounders assumed in a binding model, or others such as those set forth in U.S. Pat. App. Ser. No. 63/254,420, US Pat. App. Pub. No. 2023/0114905 A1, or Egertson et al., BioRxiv (2021), DOI: 10.1101/2021.10.11.463967, each of which is incorporated herein by reference. Adjustment of an assay can provide for more accurate identification, characterization, or quantification of macromolecules in an assay set forth herein or known in the art.

A determination of the accessibility of a macromolecule for two or more assay reagents can be used to identify non-contiguous regions of a macromolecule that interact with each other. Taking as an example a protein that is assayed using affinity reagents for two or more epitopes in the protein, an observation of reduced accessibility of two or more epitopes for the respective affinity reagents can indicate that the two or more epitopes interact with each other.

The amino acid sequence, or other structural information, for the protein can be evaluated to determine the extent to which the epitopes, and optionally their surrounding amino acids, are prone to stick together. For example, two non-polar regions may be expected to interact when the protein is in an aqueous solvent. Observation of reduced accessibility of two or more polar regions of a protein can be combined with other information about the protein, such as the polarity of amino acid sequence regions or protein domains, to determine the conformational state for at least a portion of the protein. A determination of accessibility for reactive sites in a protein can be used to confirm or refute accuracy of a structure determined for the protein using an a priori technique such as AlphaFold (Jumper et al. Nature 596, 583-589 (2021). doi.org/10.1038/s41586-021-03819-2, which is incorporated herein by reference) or other protein folding algorithm. Similarly, a determination of accessibility for reactive sites in a protein can be used to confirm or refute accuracy of a structure determined for the protein using one or more empirical technique, such as x-ray crystallography, nuclear magnetic resonance spectroscopy or biochemical assay.

A determination of the accessibility of a macromolecule for two or more assay reagents can be used to identify presence or absence of a chemical modification on the macromolecule, such as a post-translational modification on a protein. More specifically, deviation of a reactive site of a protein from expected accessibility can indicate that the reactive site is attached to a post-translationally added moiety or that the reactive site is in a splice variant region. In this situation, the protein can be assayed using a reagent that is specific for the presence or absence of the post translationally added moiety, or for the presence or absence of amino acids that occur variably in a particular splice variant. Taking the example of an an epitope in a protein that has been determined to be relatively inaccessible to a particular affinity reagent, the protein can be probed with an assay reagent that has differential reactivity to a post-translationally modified version of the epitope relative to a non-modified version of the epitope. The results of this test can distinguish whether the relative inaccessibility is due to presence of a post-translational modification on the epitope or whether inaccessibility is due to a conformational state of the protein. In some cases, the presence of a post-translational modification can be distinguished from conformationally induced inaccessibility based on the amino acid content of the epitopes evaluated. The absence of any amino acids in an epitope that are prone to post-translational modification (i.e. the epitope only includes amino acids that are inert to post-translational modification) can rule out the possibility that such a modification has occurred. Conversely, the presence of an amino acid in an epitope that is prone to a particular post-translational modification can indicate the desirability of probing the epitope for presence of the post-translational modification.

As exemplified above, a determination of reactive site accessibility can be useful for characterizing the conformational state of a given macromolecule, such as, conformation for a particular region or domain of the macromolecule. However, evaluation of reactive site accessibility on a broader scale can provide a measure of assay quality or accuracy. For example, an assay that yields higher than expected levels of inaccessible reactive sites for one or more macromolecules can be flagged or otherwise identified. The flagged assay can be continued or repeated in modified form to achieve levels of inaccessible reactive sites that are closer to expected. For example, a protein assay can be repeated using different assay reagents, such as assay reagents that target different reactive sites in one or more protein, or the protein assay can be repeated using different conditions, such as a condition that increases denaturation of the protein.

The apparent accessibility of a macromolecule to a given assay reagent can be indicative of the promiscuity of the assay reagent. For example, a first assay reagent may be relatively specific for a primary epitope, reacting with few to no secondary epitopes, whereas a second assay reagent that is specific for the primary epitope may be more promiscuous, reacting with a variety of secondary epitopes. Some configurations of the methods set forth herein can employ a control analyte to distinguish assay reagent promiscuity from characteristics of a macromolecule that impact apparent accessibility. For example, a method set forth herein can be configured to include a control in the form of relatively small molecule that displays the primary reactive site for the assay reagent. A useful control for an affinity reagent that is used to detect a particular epitope in a protein is a peptide that presents the epitope absent portions of the protein that are known or suspected of sterically blocking access of the epitope to affinity reagent.

The present disclosure provides systems that are configured to manipulate and detect macromolecules. In some cases, a system set forth herein can be configured to carry out a method set forth herein. For example, a system can be configured to detect macromolecules at single molecule resolution and determine accessibility of macromolecule structures from the results. A system can include, for example, a detector configured to acquire signals from an assay carried out between macromolecules and assay reagents. Optionally, the system can include a fluidics apparatus that is configured to contact macromolecules with assay reagents. As a further option a system can include a computer configured to perform an algorithm set forth herein. Exemplary systems are set forth in further detail below. Those skilled in the art will recognize from the present disclosure that components of the exemplary systems can be modified, replaced or combined in other ways, for example, to perform a desired function or method.

In a particular configuration, a system for determining accessibility of macromolecule structures can include (a) a detector configured to acquire signals from an array of macromolecules contacted with a plurality of different assay reagents, wherein individual addresses of the array are each attached to a single macromolecule, the macromolecule having a plurality of different reactive sites, and wherein the different assay reagents are different with respect to specificity for the reactive sites; (b) a computer processor configured to receive signals from the detector, wherein the signals are resolved with respect to the individual addresses of the array, and wherein the signals are resolved with respect to the different assay reagents, and: (i) determine a first observed reaction extent from the received signals, the first observed reaction extent configured as the fraction of the individual addresses observed to react with a first assay reagent, (ii) determine a second observed reaction extent from the received signals, the second observed reaction extent configured as the fraction of the individual addresses observed to react with a second assay reagent, (iii) determine an observed double reaction extent from the received signals, the second observed reaction extent configured as the fraction of the individual addresses observed to react with both the first assay reagent and the second assay reagent, (iv) determine an expected double reaction extent from the first observed reaction extent and the second observed reaction extent; and (v) determine accessibility of a first reactive site of the plurality of different reactive sites based on a comparison of the observed double reaction extent and the expected double reaction extent.

In another configuration, a system for determining accessibility of macromolecule structures can include (a) a detector configured to acquire signals from an array of macromolecules contacted with a plurality of different assay reagents, wherein individual addresses of the array are each attached to a single macromolecule, the macromolecule having a plurality of different reactive sites, and wherein the different assay reagents are different with respect to specificity for the reactive sites; (b) a computer processor configured to receive signals from the detector, wherein the signals are resolved with respect to the individual addresses of the array, and wherein the signals are resolved with respect to the different assay reagents, and: (i) receive candidate outcome profiles including a probability of a given candidate macromolecule reacting with the different assay reagents, (ii) compare the empirical outcome profile for each of the addresses with a plurality of the candidate outcome profiles, thereby identifying a set of addresses having a similar candidate macromolecule; (iii) identify at least two subsets of the addresses having the similar candidate macromolecule, wherein the two subsets include different isoforms of the similar candidate macromolecule; and (iv) evaluate reaction outcomes for the different isoforms to determine differential accessibility of a first reactive site in the different isoforms of the similar candidate macromolecule.

A detection system can include a detector, such as those known in the art for detecting a label or analyte set forth herein. A detector can be configured to collect signals (e.g. optical signals) from an array or other vessel containing extant proteins or other analytes. A camera such as a complementary metal-oxide-semiconductor (CMOS) or charge-coupled device (CCD) camera can be particularly useful, for example, to detect optical labels such as luminophores. The detection system can further include an excitation source configured to excite extant proteins, affinity reagents or other analytes, for example, in an array or other vessel. A detection system can include a scanning mechanism configured to effect relative movement between a detector and an array or other vessel containing extant proteins. Optionally, the scanning mechanism can be configured for time-delayed integration. Detectors that are capable of resolving proteins on an array surface including, for example, at single-molecule resolution can be particularly useful. Detectors used in DNA sequencing systems can be modified for use in a detection system or other apparatus set forth herein. Exemplary detectors are described, for example, in U.S. Pat. Nos. 7,057,026; 7,329,492; 7,211,414; 7,315,019 or 7,405,281, or US Pat. App. Pub. No. 2008/0108082 A1, each of which is incorporated herein by reference.

A detection system can further include fluidics apparatus configured to contact assay reagents and macromolecules for a reaction or step of a method set forth herein. In particular embodiments, reactions occur on arrays. Any of a variety of arrays can be present in the system, such as an array set forth herein. Macromolecules that are to be detected, for example those attached to an array, can be housed in any of a variety of reaction vessels. A particularly useful reaction vessel is a flow cell. A flow cell or other vessel can be present in a system in a permanent manner or in a removable manner, for example, being removable by hand or without the use of an auxiliary tool. A flow cell or other vessel that is present in a system can have a detection window through which a detector observes one or more macromolecules (e.g. an array of macromolecules) or other analytes. For example, an optically transparent window can be used in conjunction with an optical detector such as a fluorimeter or luminescence detector.

A fluidic apparatus can include one or more reservoirs which are fluidically connected to an inlet of a flow cell or other vessel. The reservoirs can include reagents for use in a method set forth herein. The system can further include a pump, pressure supply or other fluid displacement apparatus for driving reagents from reservoirs to the vessel. The system can include a waste reservoir that is fluidically connected to an egress of a vessel to remove spent reagents. Taking as an example an embodiment where the vessel is a flow cell, reagents can be delivered to the flow cell through a flow cell ingress and then the reagents can flow through the flow cell and out the flow cell egress to a waste reservoir. Accordingly, the flow cell can be in fluidic communication with one or more reservoirs of the system. A fluidic apparatus can include at least one manifold and/or at least one valve for directing reagents from reservoirs to a vessel where detection occurs. Exemplary fluidic apparatus that can be used in a system of the present disclosure include those configured for cyclic delivery of reagents, such as those deployed in nucleic acid sequencing reactions. Exemplary fluidic apparatus are set forth in US Pat. App. Pub. Nos. 2009/0026082 A1; 2009/0127589 A1; 2010/0111768 A1; 2010/0137143 A1; or 2010/0282617 A1; or U.S. Pat. Nos. 7,329,860; 8,951,781 or 9,193,996, each of which is incorporated herein by reference.

The present disclosure provides computer systems (e.g. computer control systems) that are programmed to implement methods, algorithms or functions set forth herein. Optionally, a computer system set forth herein can be a component of a detection system. Optionally, a computer system can be programmed or otherwise configured to performance or more of: (a) receiving an input set forth herein such as a measurement outcome, binding profile, information characterizing or identifying a plurality of candidate proteins, a binding model and/or non-specific binding rates for affinity reagents; (b) determining probabilities for affinity reagents binding to candidate macromolecules, for example, based on a binding model; (c) identifying macromolecules; (d) determining observed reaction extent for one or more macromolecules each with an assay reagent, (e) determining an observed double reaction extent for one or more macromolecules each with two or more assay reagents; (f) determining expected double reaction extent from two observed reaction extents; and (g) determining accessibility of at least one reactive site of a macromolecule.

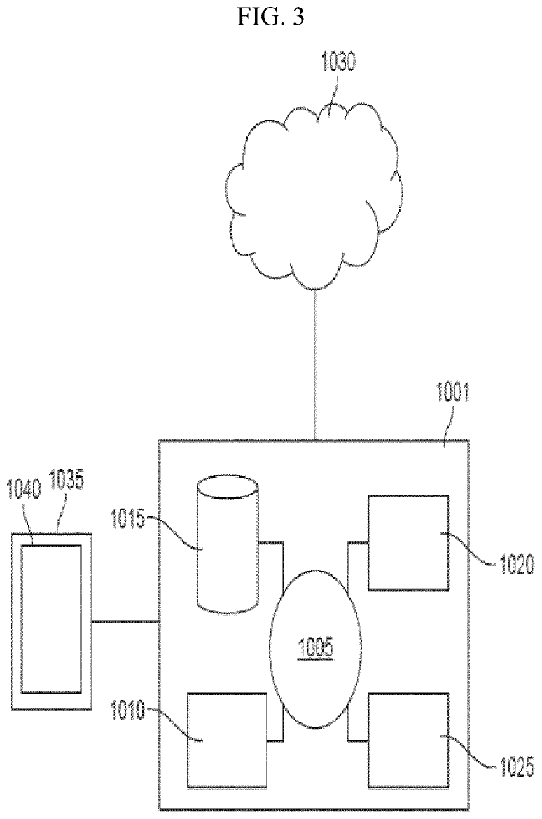

FIG. 3 shows an exemplary computer system 1001. The computer system 1001 can be an electronic device of a detection system, the electronic device being integral to the detection system or remotely located with respect to the detection system. For example, the electronic device can be a mobile electronic device. The computer system 1001 includes a computer processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet. The network 1030 in some cases is a telecommunication and/or data network. The network 1030 can include one or more computer servers, which can enable distributed computing, such as cloud computing. For example, one or more computer servers may enable cloud computing over the network 1030 (“the cloud”) to perform various aspects of analysis, calculation, and generation of the present disclosure, such as, for example, receiving information of empirical measurements of extant proteins in a sample; processing information of empirical measurements against a database comprising a plurality of protein sequences corresponding to candidate proteins, for example, using a binding model or function set forth herein; generating probabilities of a candidate protein generating empirical measurements, and/or generating probabilities that extant proteins are correctly identified in the sample. Such cloud computing may be provided by cloud computing platforms such as, for example, Amazon Web Services (AWS), Microsoft Azure, Google Cloud Platform, and IBM cloud. The network 1030, in some cases with the aid of the computer system 1001, can implement a peer-to-peer network, which may enable devices coupled to the computer system 1001 to behave as a client or a server.

The CPU 1005 can execute a sequence of machine-readable instructions, which can be embodied in a program or software. The instructions may be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and writeback.

The CPU 1005 can be part of a circuit, such as an integrated circuit. One or more other components of the system 1001 can be included in the circuit. In some cases, the circuit is an application specific integrated circuit (ASIC).

The storage unit 1015 can store files, such as drivers, libraries and saved programs. The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1001 via the network 1030.

Methods as described herein can be implemented by way of machine (e.g., computer processor) executable code stored on an electronic storage location of the computer system 1001, such as, for example, on the memory 1010 or electronic storage unit 1015. The machine executable or machine readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can include or be in communication with an electronic display 1035 that comprises a user interface (UI) 1040 for providing, for example, user selection of algorithms, binding measurement data, candidate proteins, and databases. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 1005. The algorithm can, for example, receive information of empirical measurements of macromolecules in a sample, compare information of empirical measurements against a database comprising information characteristic of macromolecules (such as candidate proteins), generate probabilities of a candidate protein generating an observed measurement outcome set, identify macromolecules, quantify macromolecules; determine observed reaction extent for one or more macromolecules each with an assay reagent, determine an observed double reaction extent for one or more macromolecules each with two or more assay reagents; determine expected double reaction extent from two observed reaction extents; and determine accessibility of at least one reactive site of a macromolecule.

The present disclosure provides a non-transitory information-recording medium that has, encoded thereon, instructions for the execution of one or more steps of the methods set forth herein, for example, when these instructions are executed by an electronic computer in a non-abstract manner. This disclosure further provides a computer processor (i.e. not a human mind) configured to implement, in a non-abstract manner, one or more of the methods set forth herein. All methods, compositions, devices and systems set forth herein will be understood to be implementable in physical, tangible and non-abstract form. The claims are intended to encompass physical, tangible and non-abstract subject matter. Explicit limitation of any claim to physical, tangible and non-abstract subject matter, will be understood to limit the claim to cover only non-abstract subject matter, when taken as a whole. Reference to “non-abstract” subject matter excludes and is distinct from “abstract” subject matter as interpreted by controlling precedent of the U.S. Supreme Court and the United States Court of Appeals for the Federal Circuit as of the priority date of this application.

### Example I

### Method of Identifying Inaccessible Regions in Proteins

This example describes methods that can be used to identify regions in a protein sequence that are inaccessible to affinity reagents. The methods are exemplified in the context of identifying proteoforms produced by differential proteolytic cleavage of a protein. The methods can be also extended to other types of proteoforms to which affinity reagents bind differentially, for example, due to presence and absence of post-translational modifications.

A proteome sample is obtained from human tissue and attached to an array. The array includes a plurality of addresses, each address being attached to one, and only one, extant protein from the sample. The array is probed with a plurality of different affinity reagents. The affinity reagents target amino acid trimer epitopes. The affinity reagents are promiscuous, binding to the trimer epitopes in a variety of amino acid sequence contexts. Individual affinity reagents may also be promiscuous with respect to binding to two or more different trimer epitopes such as those having one or more biosimilar amino acid. Empirical binding measurements from each address in the array are combined to form an empirical binding profile for the extant protein at the respective address. The empirical binding profiles are decoded with respect to candidate binding profiles for a plurality of candidate proteins that are suspected of being present in the human proteome. The extant proteins in the array are identified based on the decoding results. The samples are obtained, attached to an array, assayed with affinity reagents, and decoded as set forth in US Pat. App. Pub. No. 2023/0114905 A1, or Egertson et al., BioRxiv (2021), DOI: 10.1101/2021.10.11.463967, each of which is incorporated herein by reference.

Once the protein identifications are obtained, the accessibility of epitopes in the proteins is evaluated as follows.

1. Identify Substructures in the Data Acquired from all Addresses Identified as Having the Same Protein.

The addresses of the assayed array are binned according to the identity of the extant proteins at the respective addresses. For example, all addresses identified as having the PARP1 (poly(ADP-ribose) polymerase 1) protein are binned together. The binned addresses are then evaluated to identify substructures in the empirical binding profiles for the addresses or in the collection of likelihoods (or probabilities) that the addresses contain the identified protein.

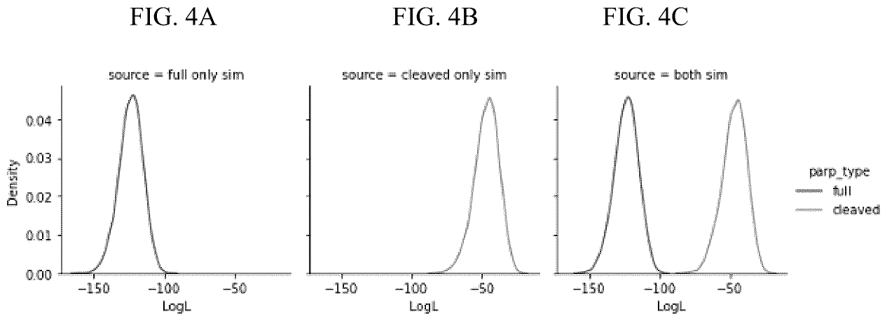

A simulated decoding of addresses having the PARP1 protein in either full length form or cleaved form was performed. The affinity reagents included those targeting epitopes found in both proteoforms and those targeting epitopes found in only the full length proteoform. The same matrix of positive binding outcomes and negative binding outcomes was used to decode full length PARP1 and cleaved PARP1. A density histogram of the logarithm of the likelihood of a PARP1 identification for the addresses was plotted. FIG. 4A shows the results of simulation for full PARP1; FIG. 4B shows the results of simulation for cleaved PARP1, and FIG. 4C shows the results for both full length and cleaved PARP1. The distributions were different since the same probabilities were used to decode data corresponding to two different proteoforms and since the affinity reagents differentially targeted the two proteoforms. Such differences can be distinguished using unsupervised learning methods such as k-means clustering.

### 2. Represent the Identified Protein as an Ordered Sequence of Epitopes

The sequence of the PARP1 protein is represented as a sequence of trimer epitopes. Trimers are used because the affinity reagents are directed to trimer epitopes. By way of example, the first 9 amino acids of PARP1 (MAESSDKLY) would yield the list of epitopes shown in Table 1.

TABLE 1
Ordered list of trimer epitopes for
amino acid sequence: MAESSDKLY

| Epitope order | Epitope |
|---|---|
| 1 | MAE |
| 2 | AES |
| 3 | ESS |
| 4 | SSD |
| 5 | SDK |
| 6 | DKL |
| 7 | KLY |

3. Calculate the Probability that Empirical Binding Measurements Obtained for Each Affinity Reagent at Each Address Correspond to Each Epitope in the Ordered Sequence of Epitopes

Exemplary calculations are shown in Table 2. Seven exemplary epitopes of PARP1 are listed in the second column and the probability of each of four different affinity reagents (AR1, AR2, AR3 and AR4) are listed in columns three through six. In this example, AR1 and AR3 are indicated as having produced a positive binding outcome at the address (“+”), whereas AR2 and AR4 are indicated as having produced negative binding outcomes at the address (“−”). The sum of probabilities for all affinity reagents binding to each epitope is calculated as shown in the seventh column (Sum(all_ARs)). For column eight, the probabilities for each reagent binding to each epitope is multiplied by 1 or 0, depending upon observation of a positive binding outcome or negative binding outcome, respectively, and then the products of the multiplication are summed (Sum(all_ARs*bind)).

TABLE 2
Binding Probability Calculations for One Address

| Epitope | | AR1 | AR2 | AR3 | AR4 | | |
|---|---|---|---|---|---|---|---|
| order | Epitope | (+) | (−) | (+) | (−) | Sum(all_ARs) | Sum(all_ARs * bind) |
| 1 | MAE | 0.17 | 0.18 | 0.24 | 0.17 | 0.76 | 0.35 |
| 2 | AES | 0.21 | 0.17 | 0.15 | 0.14 | 0.67 | 0.31 |
| 3 | ESS | 0.23 | 0.14 | 0.04 | 0 | 0.41 | 0.14 |
| 4 | SSD | 0.12 | 0.23 | 0.21 | 0.21 | 0.77 | 0.44 |
| 5 | SDK | 0.23 | 0.23 | 0.12 | 0 | 0.58 | 0.23 |
| 6 | DKL | 0.16 | 0.08 | 0.07 | 0.23 | 0.54 | 0.31 |
| 7 | KLY | 0.13 | 0.13 | 0.19 | 0.03 | 0.45 | 0.16 |

4. Identify Regions of Unlikely Consecutive Non-Binding of Affinity Reagents from the Probabilities Calculated for Each Address in Step 3

The probability of each affinity reagent yielding a negative binding outcome for each epitope is calculated. For example, the probability can be calculated as 1−(Sum(all_ARs*bind)). In this example, subsequence length is chosen, and the values of 1−(Sum(all_ARs*bind)) for all epitopes in the subsequence are multiplied to obtain a probability of no positive binding outcomes being observed for the address. The last column of Table 3 shows the values of 1−(Sum(all_ARs*bind)) for a subsequence including the first seven epitopes in the sequence of PARP1. Multiplying the values in the last column gives a 0.0964 probability of no positive binding outcomes being observed for the address.

TABLE 3
Non-Binding Probability Calculations for One Address

| Epitope | | | Sum(all_Ars * | 1 − Sum(all_ARs * |
|---|---|---|---|---|
| order | Epitope | Sum(all_ARs) | bind) | bind) |
| 1 | MAE | 0.76 | 0.35 | 0.65 |
| 2 | AES | 0.67 | 0.31 | 0.69 |
| 3 | ESS | 0.41 | 0.14 | 0.86 |
| 4 | SSD | 0.77 | 0.44 | 0.56 |
| 5 | SDK | 0.58 | 0.23 | 0.77 |
| 6 | DKL | 0.54 | 0.31 | 0.69 |
| 7 | KLY | 0.45 | 0.16 | 0.84 |

### 5. Generate a Null Distribution

A within-protein-test can be used, wherein an unsupervised learning method classifies PARP1 as including two or more proteoforms. Regions of nonbinding are compared using one group of epitopes as a null distribution (assuming another group has the region of nonbinding).

Alternatively, a within-address-test can be used, wherein a permutation test is performed by shuffling the positive binding outcomes and negative binding outcomes within a given address.

In another option, the empirical results for addresses assigned to PARP1 can be compared to a random sampling of remaining addresses observed in the assay from which the PARP1 results were obtained.

In yet another option, the empirical results for addresses assigned to PARP1 can be compared to the results of a simulated PrISM experiment in which there are no assumed regions of inaccessibility or proteoforms. See US Pat. App. Pub. No. 2023/0114905 A1, or Egertson et al., BioRxiv (2021), DOI: 10.1101/2021.10.11.463967 (each of which is incorporated herein by reference) for a description of PrISM experiments.

### 6. Compare Null Distribution to the Results of Step 4

The confidence (p-value) of the presence of a region of inaccessibility or two or more proteoforms is calculated by comparing the result to the set of permutations performed as set forth above. The set of permutations serve as a null distribution. The probability of a false positive is calculated by dividing the number of permutations that out-perform the initial result by the total number of permutations.

In the event there are many proteoforms, the confidence values (p-values) can be corrected for multiple hypothesis testing. Some useful methods for this include Benjamini-Hochburg correction, Bonferroni correction, and the method of Storey and Tibshirani.

What is claimed is:

1. A method of determining accessibility of macromolecule structures, comprising:
(a) contacting an array of macromolecules with a plurality of different assay reagents, wherein individual addresses of the array are each attached to a single macromolecule, the macromolecule comprising a plurality of different reactive sites, and wherein the different assay reagents are different with respect to specificity for the reactive sites;
(b) detecting reaction of the array of macromolecules with the different assay reagents, whereby the individual addresses of the array are resolved, and whereby the different assay reagents are resolved;
(c) determining a first observed reaction extent comprising the fraction of the individual addresses observed to react with a first assay reagent in step (b);
(d) determining a second observed reaction extent comprising the fraction of the individual addresses observed to react with a second assay reagent in step (b);
(e) determining an observed double reaction extent comprising the fraction of the individual addresses observed to react with both the first assay reagent and the second assay reagent in step (b);
(f) determining an expected double reaction extent from the first observed reaction extent and the second observed reaction extent; and
(g) determining accessibility of a first reactive site of the plurality of different reactive sites based on a comparison of the observed double reaction extent and the expected double reaction extent.

2. The method of claim 1, wherein the macromolecules comprise proteins.

3. The method of claim 1, wherein the macromolecules have a denatured conformation in steps (a) and (b).

4. The method of claim 1, wherein the macromolecules have a native conformation in steps (a) and (b).

5. The method of claim 1, wherein the macromolecules have a molecular domain in common.

6. The method of claim 5, wherein the macromolecules have the same molecular composition.

7. The method of claim 1, wherein the assay reagents comprise affinity reagents, the reaction comprises binding of the affinity reagents to macromolecules of the array, the reactive sites comprise epitopes and the reaction extents comprise binding extents.

8. The method of claim 1, wherein the assay reagents comprise labelling reagents, the reaction comprises labelling the macromolecules of the array with the labelling reagents, the reactive sites comprise moieties that react with the labelling reagents and the reaction extents comprise labelling extents.

9. The method of claim 1, wherein the assay reagents comprise cleavage reagents, the reaction comprises cleaving the macromolecules of the array, the reactive sites comprise linkages that are cleaved by the cleavage reagents and the reaction extents comprise cleavage extents.

10. The method of claim 1, wherein the assay reagents comprise an enzyme, the reaction is catalyzed by the enzyme, the reactive sites comprise moieties that are covalently modified via catalytic activity of the enzyme and the reaction extents comprise modification extents.

11. The method of claim 1, wherein the comparison of the observed double reaction extent and the expected double reaction extent in step (g) comprises a ratio of the expected double reaction extent to the observed double reaction rate.

12. The method of claim 1, wherein step (g) comprises determining accessibility of the first reactive site relative to accessibility of a second reactive site of the plurality of different reactive sites based on a comparison of the observed double reaction extent and the expected double reaction rate.

13. The method of claim 1, wherein step (g) comprises determining relative accessibility for the first reactive site compared to accessibility of at least two other reactive sites of the plurality of different reactive sites.

14. The method of claim 1, wherein the first assay reagent and second assay reagent are separately contacted with the array of macromolecules.

15. The method of claim 14, wherein the first assay reagent is removed from the array of macromolecules prior to the detecting of the reaction of the array of macromolecules with the second assay reagent.

16. A system for determining accessibility of macromolecule structures, comprising
(a) a detector configured to acquire signals from an array of macromolecules contacted with a plurality of different assay reagents, wherein individual addresses of the array are each attached to a single macromolecule, the macromolecule comprising a plurality of different reactive sites, and wherein the different assay reagents are different with respect to specificity for the reactive sites;
(b) a computer processor configured to receive signals from the detector, wherein the signals are resolved with respect to the individual addresses of the array, and wherein the signals are resolved with respect to the different assay reagents, and:
(i) determine a first observed reaction extent from the received signals, the first observed reaction extent comprising the fraction of the individual addresses observed to react with a first assay reagent,
(ii) determine a second observed reaction extent from the received signals, the second observed reaction extent comprising the fraction of the individual addresses observed to react with a second assay reagent,
(iii) determine an observed double reaction extent from the received signals, the second observed reaction extent comprising the fraction of the individual addresses observed to react with both the first assay reagent and the second assay reagent,
(iv) determine an expected double reaction extent from the first observed reaction extent and the second observed reaction extent; and
(v) determine accessibility of a first reactive site of the plurality of different reactive sites based on a comparison of the observed double reaction extent and the expected double reaction extent.

17. A method of determining accessibility of macromolecule structures, comprising
(a) contacting an array of macromolecules with a plurality of different assay reagents, wherein individual addresses of the array are each attached to a single macromolecule, the macromolecule comprising a plurality of different reactive sites, and wherein the different assay reagents are different with respect to specificity for the reactive sites;
(b) detecting reaction of the array of macromolecules with the different assay reagents, whereby the individual addresses of the array are resolved, and whereby the different assay reagents are resolved;
(c) determining a first observed reaction extent comprising the fraction of the individual addresses having a first reactive site that is observed to react with a first assay reagent of the plurality of different assay reagents in step (b);
(d) providing an expected reaction extent comprising the extent to which the assay reagent reacts with a candidate macromolecule having the same molecular structure as the macromolecules at the fraction of the individual addresses observed to react with the first assay reagent in step (b);
(e) determining accessibility of the first reactive site of the macromolecules based on a comparison of the observed reaction extent and the expected reaction extent.

18. A method of determining accessibility of macromolecule structures, comprising
(a) contacting an array of macromolecules with a plurality of different assay reagents, wherein individual addresses of the array are each attached to a single macromolecule, the macromolecule comprising a plurality of different reactive sites, and wherein the different assay reagents are different with respect to specificity for the reactive sites;
(b) detecting reaction outcomes for individual addresses in the array with each of the different assay reagents, whereby the individual addresses of the array are resolved, whereby the different assay reagents are resolved, and wherein a collection of reaction outcomes for each address comprises an empirical outcome profile;
(c) comparing the empirical outcome profiles for the addresses with a plurality of candidate outcome profiles, each of the candidate outcome profiles comprising a probability of a given candidate macromolecule reacting with the different assay reagents, thereby identifying a set of addresses having a similar candidate macromolecule;
(d) identifying two subsets of the addresses having the similar candidate macromolecule, wherein the two subsets comprise different isoforms of the similar candidate macromolecule; and
(e) evaluating reaction outcomes for the different isoforms to determine differential accessibility of a first reactive site in the different isoforms of the similar candidate macromolecule.
